(19) United States
(12) Patent Application Publication
(10) Pub. No.: US 2025/0213585 A1
(43) Pub. Date: Jul. 3, 2025

(54) COMPOSITIONS AND METHODS OF A CONCOMITANT THERAPY OF ALTERNATING ELECTRIC FIELDS AND NOTCH-1 INHIBITOR

(71) Applicant: NOVOCURE GMBH, Root D4 (CH)

(72) Inventors: Jiang Hai, Shanghai (CN); Dongxiao Zhuang, Shanghai (CN); Pengjie Hong, Shanghai (CN)

(21) Appl. No.: 19/003,041

(22) Filed: Dec. 27, 2024

Related U.S. Application Data
(60) Provisional application No. 63/615,418, filed on Dec. 28, 2023.
(60) Provisional application No. 63/726,002, filed on Nov. 27, 2024.

(51) Int. Cl. A61K 31/55; A61N 1/36; A61P 35/00

(52) U.S. Cl. CPC A61K 31/55; A61N 1/36002; A61P 35/00

(57) ABSTRACT

Disclosed are methods of treating a subject in need thereof comprising applying an alternating electric field, to a target site of the subject in need thereof; and administering a a Notch1 inhibitor or Hes5 inhibitor to the subject in need thereof. Disclosed are methods of preventing cancer recurrence in a subject having cancer comprising: applying an alternating electric field to a target site of the subject in need thereof; and administering a Notch1 inhibitor or Hes5 inhibitor to the subject in need thereof. Disclosed are methods of reducing cancer cell growth comprising: applying an alternating electric field to a population of cells comprising one or more cancer cells; and contacting a Notch1 inhibitor or Hes5 inhibitor to the population of cells. Disclosed are methods of sensitizing cancer cells to an alternating electric field comprising: applying an alternating electric field to a population of cells comprising one or more cancer cells; and contacting a Notch1 inhibitor or Hes5 inhibitor to the population of cells.

### CROSS-REFERENCE TO RELATED APPLICATIONS

This application claims the benefit of U.S. Provisional Patent Application No. 63/615,418, filed Dec. 28, 2023 and U.S. Provisional Patent Application No. 63/726,002, filed Nov. 27, 2024, each of which is incorporated by reference herein in its entirety.

### BACKGROUND

Neurogenic locus notch homolog protein 1 (Notch 1) is a protein encoded in humans by the NOTCH1 gene. Notch 1 is a single-pass transmembrane receptor. Notch family members play a role in a variety of developmental processes by controlling cell fate decisions. The Notch signaling network is an evolutionarily conserved intercellular signaling pathway that regulates interactions between physically adjacent cells. The Notch signaling pathway is a regulator of self-renewal and differentiation in several tissues and cell types. Notch is a binary cell-fate determinant, and its hyperactivation has been implicated as oncogenic in several cancers including breast cancer and T-cell acute lymphoblastic leukemia (T-ALL).

The protein produced from the NOTCH1 gene has such diverse functions that the gene is considered both an oncogene and a tumor suppressor. Oncogenes typically promote cell proliferation or survival, and when mutated, they have the potential to cause normal cells to become cancerous. In contrast, tumor suppressors keep cells from growing and dividing too fast or in an uncontrolled way, preventing the development of cancer; mutations that impair tumor suppressors can lead to cancer development.

The direct effects of Notch inhibition on cancer cells may vary. Given the interaction of Notch with important anti-apoptotic pathways such as Akt, it is perhaps not surprising that Notch inhibition has most frequently been shown to trigger apoptosis in cancer cells.

Notch inhibition as cancer therapy may also pose significant risks to subjects such as damage to normal stem cells in the body or other toxicities. Several independent studies have discovered that Notch1 overexpression is correlated with tumor progression, poor prognosis, and proliferation of lung cancer cell lines. While pre-clinical and clinical work has been done in attempts to bring Notch1 inhibitors to the clinic, to date none have been FDA approved.

What is needed are therapies that overcome the failures of Notch1 inhibitors to treat cancer or reduce Notch1 inhibitor side effects. What is also needed are therapies and therapeutic approaches to overcome tumor recurrence after prolonged alternating electric field therapy.

### BRIEF SUMMARY

Alternating electric fields are shown herein to induce expression of Notch1. In addition, the data provided herein evidences that disrupting the NOTCH1 gene or inhibiting Notch1 protein can increase sensitivity to an alternating electric field. Due to the potential negative effects Notch1 can have on cancer cells, the current invention is directed to concomitant therapy of alternating electric fields and Notch1 inhibitors.

Additionally, alternating electric fields can induce the degradation of Notch1, while cellular Notch1 mRNA expression levels are elevated. Thus, described herein are methods and compositions directed to concomitant therapy of alternating electric fields and Notch1 inhibitors to increase the efficacy of alternating electric fields or sensitivity of a subject or cell to alternating electric fields. It was surprising that administering a Notch1 inhibitor to a subject or exposing a cell to a Notch1 inhibitor increased the efficacy of an alternating electric fields on the subject or cell. Furthermore, tumor recurrence after prolonged therapy with alternating electric fields could be avoided/overcome by administering a Notch1 inhibitor.

Disclosed are methods of treating a subject in need thereof comprising: applying an alternating electric field to a target site of the subject in need thereof; and administering a Notch1 inhibitor to the subject in need thereof.

Disclosed are methods of preventing cancer recurrence in a subject having cancer comprising: applying an alternating electric field to a target site of the subject in need thereof; and administering a Notch1 inhibitor to the subject in need thereof.

Disclosed are methods of reducing cancer cell growth comprising: applying an alternating electric field to a population of cells comprising one or more cancer cells; and contacting a Notch1 inhibitor to the population of cells.

Disclosed are methods of sensitizing cancer cells to an alternating electric field comprising: applying an alternating electric field to a population of cells comprising one or more cancer cells; and contacting a Notch1 inhibitor to the population of cells.

Additional advantages of the disclosed methods and compositions will be set forth in part in the description which follows, and in part will be understood from the description, or may be learned by practice of the disclosed methods and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIGS. 1A-C show the results of a CRISPR/cas9 library screen. FIGS. 1A and 1B shows that cells were reduced to around 20% of their initial survival rate after an initial exposure to an alternating electric field after the cells were allowed to recover to 100% survival. FIG. 1C shows that when the Notch1 gene is disrupted, the cells may exhibit increased sensitivity to an alternating electric field.

FIG. 2 shows that expression levels of Notch1 mRNA increased in varying degrees in different ell types upon extended duration of alternating electric field treatment.

FIGS. 3A-C show that alternating electric fields can lead to a degradation of Notch1 protein levels.

FIG. 4 shows that the combination of RO4929097 with an alternating electric field sensitizes alternating electric field therapy and degradation of Notch1 protein during low-intensity, long duration alternating electric field therapy.

FIG. 5 shows that treatment with a Notch1 inhibitor exhibited no significant impact on colonogenic abilities following re-seeding after treatment; however, when the Notch1 inhibitor was combined with an alternating electric field, a sensitization effect in the cells was observed.

FIGS. 6A-D show glioblastoma cell lines U251 and U87 treated with TTFields, Notch1 inhibitor, or a combination of TTFields plus Notch1 inhibitor.

FIGS. 7A-D shows there was no effect of a Notch1 inhibitor when used in combination with temozolomide (TMZ).

FIG. 8 shows the combination of an alternating electric field with DAPT enhances the therapeutic efficacy of the alternating electric field.

FIGS. 9A-9F show 200 kHz and 350 kHz are particularly potent frequencies in eliciting death in Eu-Myc p19 Arf−/− cells; 9D shows CRISPR screen results in which sg RNA against Notch was depleted, indicating that Notch knockout sensitizes cells to TTFields . . . 9E and 9F shows that upon TTFields treatment, Notch mRNA level was upregulated.

FIGS. 10A-10G show Notch inhibitors RO enhances growth suppression by TTFields. 10E: Notch inhibitor RO does not enhance killing by chemotherapeutic drug TMZ (temozolomide). 10F-G: shRNA knockdown of Notch enhances growth suppression by TTFields.

FIGS. 11A-11H show TTFields treatment leads to decrease of Notch protein level despite increase in Notch mRNA level. 11E-H, TTFields treatment leads to Notch degradation through lysosomal pathway, but had no effects on several other plasma membrane proteins including TNFR and LAG3

FIGS. 12A-12E show TTFields treatment reduced protein level of stemness markers such as Notch1, Nestin, Sox2 and CD133.

FIGS. 13A-13I show in patient derived GBM organoid, TTFields treatment reduced protein level of stemness markers such as Notch1, Nestin, Sox2 and CD133

FIGS. 14A-14I show clinical data on TTFields-treated GBM patient demonstrating Notch1 as a determinant of TTFields treatment outcome.

### DETAILED DESCRIPTION

The disclosed methods and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a Notch1 inhibitor” includes a plurality of such Notch1 inhibitors, reference to “the Notch1 inhibitor” is a reference to one or more Notch1 inhibitors and equivalents thereof known to those skilled in the art, and so forth.

As used herein, a “target site” is a specific site or location within or present on a subject or patient. For example, a “target site” can refer to, but is not limited to a cell (e.g., a cancer cell or fibroblast), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the brain, ovaries, lungs, breast, thymus, kidney, digestive organs, skin, pancreas, thyroid, liver, stomach, uterus, or colon. In some aspects, a cell or population of cells that can be a target site or a target cell include, but are not limited to, a cancer cell (e.g., a brain cancer cell, ovarian cancer cell, breast cancer cell, thyroid cancer cell, kidney cancer cell, lung cancer cell, digestive organ cancer cell, colon cancer cell, or endometrial cancer cell). In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electric field delivered to a subject, a sample obtained from a subject or to a specific location within a subject or patient (e.g., a target site such as a cell). In some aspects, the alternating electric field can be in a single direction or multiple directional, e.g., alternate directions across the target site. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and is described in U.S. Pat. No. 7,565,205, which is incorporated herein by reference in its entirety for its teaching of TTFields.

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. “Subject” can be used interchangeably with “individual” or “patient.” For example, the subject of administration can mean the recipient of the alternating electric field. For example, the subject of administration can be a subject with cancer, e.g., glioblastoma, ovarian cancer, non-small cell lung cancer, breast cancer (e.g. triple-negative breast cancer), papillary Thyroid cancer, clear cell renal cell carcinoma, hepatocellular cancer, pleural mesothelioma, pancreatic cancer, lung cancer, gastric cancer, colon cancer or endometrial carcinoma.

By “treat” is meant to administer or apply a therapeutic, such as alternating electric fields and a Notch1 inhibitor, to a subject, such as a human or other mammal (for example, an animal model), that has cancer or has an increased susceptibility for developing cancer, in order to prevent or delay a worsening of the effects of the disease or infection, or to partially or fully reverse the effects of cancer. For example, treating a subject having glioblastoma can comprise delivering a therapeutic to a cell in the subject.

By “prevent” is meant to minimize or decrease the chance that a subject develops cancer.

As used herein, the terms “administering” and “administration” refer to any method of providing a Notch1 inhibitor to a subject directly or indirectly to a target site. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat cancer. In further various aspects, a preparation can be administered prophylactically; that is, administered for prevention of cancer. In an aspect, the skilled person can determine an efficacious dose, an efficacious schedule, or an efficacious route of administration so as to treat a subject. In some aspects, administering comprises exposing or applying. Thus, in some aspects, exposing a target site or subject to alternating electrical fields or applying alternating electrical fields to a target site or subject means administering alternating electrical fields to the target site or subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise applying alternating electric fields. In some aspects, the alternating electric field used in the methods disclosed herein can be applied at a frequency for a period of time. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. In some aspects, the frequency of the alternating electric field is between 50 kHz and 1 MHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 150 and 300 kHz, between 180 and 220 kHz, or between 210 and 400 kHz. In some aspects, the frequency of the alternating electric fields can be about 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 150 or 250 kHz, 200 kHz to about 400 kHz, from about 250 kHz to about 350 kHz, and may be around 300 kHz.

In some aspects, the field strength of the alternating electric field can be between 0.5 and 10 V/cm RMS. In some aspects, the field strength of the alternating electric field can be between 1 and 4 V/cm RMS. In some aspects, different field strengths can be used (e.g., between 0.1 and 10 V/cm RMS). In some aspects, the field strength can be about 1.75 V/cm RMS. In some embodiments the field strength is at least 1 V/cm RMS. In some aspects, the field strength can be about 0.9 V/cm RMS. In other embodiments, combinations of field strengths are applied, for example combining two or more frequencies at the same time, and/or applying two or more frequencies at different times.

In some aspects, the alternating electric field can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric field can be applied every day for a two hour duration. For example, the alternating electric field can be applied for at least 4 hours per day, at least 8 hours per day, at least 12 hours per day, at least 16 hours per day, or at least 20 hours per day. In some aspects, the alternating electric field can be applied for at least 4, 8, 12, 16, or 20 hours per day for at least 2 days. In some aspects, the alternating electric field can be applied for at least 4, 8, 12, 16, or 20 hours per day for at least 3 days. In some aspects, the alternating electric fields can be applied for at least 4, 8, 12, 16, or 20 hours per day for at least 7 days.

In some aspects, the consecutive exposure may last for at least 6 hours, at least 12 hours, at least 24 hours, at least 36 hours, at least 48 hours, or at least 72 hours or more.

In some aspects, the cumulative exposure may last for at least 42 hours, at least 84 hours, at least 168 hours, at least 250 hours, at least 400 hours, at least 500 hours, at least 750 hours, or more.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

### C. Methods of Treating

Disclosed are methods of treating a subject in need thereof comprising applying an alternating electric field to a target site of the subject in need thereof; and administering a Notch1 inhibitor to the subject in need thereof. In some aspects, the alternating electric field is administered at a frequency for a period of time.

In some aspects, the Notch1 inhibitor is a γ Secretase Inhibitor. In some aspects, the Y Secretase Inhibitor is DAPT (GSI-IX), LY-411575, LY900009, RO4929097 (RG-4733), YO-01027 (Dibenzazepine), Crenigacestat (LY3039478), Semagacestat, BMS-906024, Avagacestat (BMS-708163), Sulindac sulfide, Dihydroergocristine mesylate (DHEC mesylate), γ-Secretase-IN-1, BMS 433796, L-685458, BMS 299897, MDL-28170, Nirogacestat, BT-GSI, E 2012, MK-0752, SPL-707, MRK-560, Itanapraced, JNJ-40418677, Z-Ile-Leu-aldehyde (Z-IL-CHO), or BT-GSI.

In some aspects, the Notch1 inhibitor is Jagged-1 (188-204) TFA, JI130, Procyanidin B2, 3,3′-di-O-gallate, Limantrafin (CB-103), Tangeretin (Tangeritin), Carvacrol, RBPJ Inhibitor-1 (RIN1), IMR-1, Psoralidin, BMS-906024 or Tarlatamab (AMG-757), Bruceine D, FLI-06, ZLDI-8, JI051, Rovalpituzumab, BMS-983970, BMS-986115, Brontictuzumab, IMR-1A, Tarextumab, Demcizumab, ASR-490, Navicixizumab, SAHM1, SAHM1 TFA, Enoticumab, FLI-06, SAHM1, NVS-ZP7-4, or JI051.

In some aspects, the Notch1 inhibitor is an anti-Notch1 antibody (mAb to DSL ligands, NRR antibodies, mAbs to Notch receptors, mAbs to Nicastrin, mAbs to ADAMS, mAbs to NICD, mAbs to MAM), a Notch1 decoy, Notch1 antagonist, siRNA, CRISPR/CAS, Y-secretase inhibitor, α-secretase inhibitor, acidification inhibitor, or a MAM stapled peptide.

In some aspects, the subject in need thereof has cancer. In some aspects, the cancer can be, but is not limited to, glioblastoma, ovarian cancer, non-small cell lung cancer, breast cancer (e.g. triple-negative breast cancer), papillary thyroid cancer, clear cell renal cell carcinoma, hepatocellular cancer, pleural mesothelioma, pancreatic cancer, lung cancer, gastric cancer, colon cancer or endometrial carcinoma. In some aspects, the subject does not have an autoimmune disease, a lymphoproliferative disease, a degenerative disease, small cell lung cancer (SCLC), epilepsy, bipolar disorder, metabolic disease, HIV infection, migraines, multiple myeloma, pancreatic cancer, or Alzheimer's disease.

In some aspects, the target site comprises cancer cells. In some aspects, the cancer cells can be, but are not limited to, a brain cancer cell, ovarian cancer cell, breast cancer cell, thyroid cancer cell, kidney cancer cell, lung cancer cell, digestive organ cancer cell, colon cancer cell, or endometrial cancer cell.

In some aspects, the subject has become desensitized to the alternating electric field.

In some aspects, the Notch1 inhibitor reduces cancer cell growth. In some aspects, the Notch1 inhibitor reduces cancer cell proliferation. In some aspects, the Notch1 inhibitor reduces metastasis.

In some aspects, the alternating electric field is applied before, after, or simultaneously with administering the Notch1 inhibitor. In some aspects, the step of applying the alternating electric fields begins at least one hour before a Notch1 inhibitor is administered. In some aspects, when the Notch1 inhibitor is administered before applying the alternating electric fields, the cells are surprisingly sensitized to the alternating electric fields, thereby increasing the efficacy of the alternating electric fields. In some aspects, the step of applying the alternating electric fields begins at least 30 minutes before administering a Notch1 inhibitor. In some aspects, applying the alternating electric fields simultaneously can mean applying within 5, 10, 15, 20, 25, 30, 35, 40, 45, 50, 55, or 60 minutes before or after administering an Notch1 inhibitor. In some aspects, the alternating electric fields can be applied and the Notch1 inhibitor administered at least 1, 2, 3, 4, 5, 6, 7, 8, 9, 10, 11, 12, 13, 14, 15, 16, 17, 18, 19, 20, 21, 22, 23, or 24 hours from each other.

In some aspects, the Notch1 inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

In some aspects, the frequency and/or field strength of the alternating electric field can be any of those described herein and can be applied in any of the manners described herein. In some aspects, the frequency of the alternating electric field is between 50 kHz and 1 MHz. In some aspects, the frequency of the alternating electric field is about 150 or 250 kHz. In some aspects, the alternating electric field has a field strength of between 0.5 and 10 V/cm RMS. In some aspects, the alternating electric field has a field strength of about 0.9 V/cm RMS.

In some aspects, the methods of treating further comprise administering a cancer therapeutic. In some aspects, a cancer therapeutic can be any known cancer therapeutic, such as, but not limited to, a chemotherapeutic agent or anti-inflammatory agent.

In some aspects, after applying an alternating electric field and prior to administering a Notch1 inhibitor, detecting an increase in Notch1 expression in the subject or cell. Thus, in some aspects, the method comprises only administering a Notch1 inhibitor to those subjects having an increase in Notch1 after applying an alternating electric field.

In some aspects, administering a Notch1 inhibitor is performed 1, 2, 3, 4, 5, 6, 7, 8, 9, or 10 days after applying an alternating electric field is performed. In some aspects, administering a Notch1 inhibitor is performed simultaneously with applying an alternating electric field. In some aspects, administering a Notch1 inhibitor is performed within hours, days, or weeks of applying an alternating electric field.

In some aspects, nanoparticles can be used in the disclosed methods. For example, in some aspects, chelators, such as Notch1 inhibitors, can be carried by nanoparticles (e.g. encapsulated by nanoparticles) to provide specific chelation at the target site thus avoiding systemic influence of chelating calcium. In some aspects, the nanoparticles can be induced to release the Notch1 inhibitor in the presence of alternating electric fields. For example, in some aspects, alternating electric fields can cause the nanoparticle to burst thus releasing the Notch1 inhibitor. In some aspects, a nanoparticle can be a polymeric nanoparticle, liposome, micelle, metal nanoparticles. In some aspects, the nanoparticle comprises a target site-specific targeting moiety. In some aspects, the target site-specific targeting moiety can be a cancer cell-specific targeting moiety. In some aspects, the targeting moiety can direct, or target, the nanoparticle to a specific target site. The target site-specific targeting moiety can be a chemical, compound, peptide or nucleic acid. Examples of targeting moieties include, but are not limited to, molecules that recognize receptors on specific cell types.

### D. Method of Preventing Cancer Recurrence

Disclosed are methods of preventing cancer recurrence in a subject having cancer comprising: applying an alternating electric field, to a target site of the subject in need thereof; and administering a Notch1 inhibitor to the subject in need thereof. In some aspects, the alternating electric field is administered at a frequency for a period of time.

In some aspects, the Notch1 inhibitor is a γ Secretase Inhibitor. In some aspects, the γ Secretase Inhibitor is DAPT (GSI-IX), LY-411575, LY900009, RO4929097 (RG-4733), YO-01027 (Dibenzazepine), Crenigacestat (LY3039478), Semagacestat, BMS-906024, Avagacestat (BMS-708163), Sulindac sulfide, Dihydroergocristine mesylate (DHEC mesylate), γ-Secretase-IN-1, BMS 433796, L-685458, BMS 299897, MDL-28170, Nirogacestat, BT-GSI, E 2012, MK-0752, SPL-707, MRK-560, Itanapraced, JNJ-40418677, Z-Ile-Leu-aldehyde (Z-IL-CHO), or BT-GSI.

In some aspects, the Notch1 inhibitor is Jagged-1 (188-204) TFA, JI130, Procyanidin B2, 3,3′-di-O-gallate, Limantrafin (CB-103), Tangeretin (Tangeritin), Carvacrol, RBPJ Inhibitor-1 (RIN1), IMR-1, Psoralidin, BMS-906024 or Tarlatamab (AMG-757), Bruceine D, FLI-06, ZLDI-8, JI051, Rovalpituzumab, BMS-983970, BMS-986115, Brontictuzumab, IMR-1A, Tarextumab, Demcizumab, ASR-490, Navicixizumab, SAHM1, SAHM1 TFA, Enoticumab, FLI-06, SAHM1, NVS-ZP7-4, or JI051.

In some aspects, the Notch1 inhibitor is an anti-Notch1 antibody (mAb to DSL ligands, NRR antibodies, mAbs to Notch receptors, mAbs to Nicastrin, mAbs to ADAMS, mAbs to NICD, mAbs to MAM), a Notch1 decoy, Notch1 antagonist, siRNA, CRISPR/CAS, Y-secretase inhibitor, α-secretase inhibitor, acidification inhibitor, or a MAM stapled peptide.

In some aspects, the subject in need thereof has cancer. In some aspects, the cancer can be, but is not limited to, glioblastoma, ovarian cancer, non-small cell lung cancer, breast cancer (e.g. triple-negative breast cancer), papillary thyroid cancer, clear cell renal cell carcinoma, hepatocellular cancer, pleural mesothelioma, pancreatic cancer, lung cancer, gastric cancer, colon cancer or endometrial carcinoma. In some aspects, the subject does not have an autoimmune disease, a lymphoproliferative disease, a degenerative disease, small cell lung cancer (SCLC), epilepsy, bipolar disorder, metabolic disease, HIV infection, migraines, multiple myeloma, pancreatic cancer, or Alzheimer's disease.

In some aspects, the target site comprises cancer cells. In some aspects, the cancer cells can be, but are not limited to, a brain cancer cell, ovarian cancer cell, breast cancer cell, thyroid cancer cell, kidney cancer cell, lung cancer cell, digestive organ cancer cell, colon cancer cell, or endometrial cancer cell.

In some aspects, the subject has become desensitized to the alternating electric field.

In some aspects, the Notch1 inhibitor reduces cancer cell growth. In some aspects, the Notch1 inhibitor reduces cancer cell proliferation. In some aspects, the Notch1 inhibitor reduces metastasis.

In some aspects, the alternating electric field is applied before, after, or simultaneously with administering the Notch1 inhibitor. In some aspects, the step of applying the alternating electric fields begins at least one hour before a Notch1 inhibitor is administered. In some aspects, when the Notch1 inhibitor is administered before applying the alternating electric fields, the cells are surprisingly sensitized to the alternating electric fields, thereby increasing the efficacy of the alternating electric fields. In some aspects, the step of applying the alternating electric fields begins at least 30 minutes before administering a Notch1 inhibitor. In some aspects, applying the alternating electric fields simultaneously can mean applying within 5, 10, 15, 20, 25, 30, 35, 40, 45, 50, 55, or 60 minutes before or after administering an Notch1 inhibitor. In some aspects, the alternating electric fields can be applied and the Notch1 inhibitor administered at least 1, 2, 3, 4, 5, 6, 7, 8, 9, 10, 11, 12, 13, 14, 15, 16, 17, 18, 19, 20, 21, 22, 23, or 24 hours from each other.

In some aspects, the Notch1 inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

In some aspects, the frequency and/or field strength of the alternating electric field can be any of those described herein and can be applied in any of the manners described herein. In some aspects, the frequency of the alternating electric field is between 50 kHz and 1 MHz. In some aspects, the frequency of the alternating electric field is about 150 or 250 kHz. In some aspects, the alternating electric field has a field strength of between 0.5 and 10 V/cm RMS. In some aspects, the alternating electric field has a field strength of about 0.9 V/cm RMS.

In some aspects, the methods of preventing cancer recurrence further comprise administering a cancer therapeutic. In some aspects, a cancer therapeutic can be any known cancer therapeutic, such as, but not limited to, a chemotherapeutic agent or anti-inflammatory agent.

In some aspects, after applying an alternating electric field and prior to administering a Notch1 inhibitor, detecting an increase in Notch1 expression in the subject or cell. Thus, in some aspects, the method comprises only administering a Notch1 inhibitor to those subjects having an increase in Notch1 after applying an alternating electric field.

In some aspects, administering a Notch1 inhibitor is performed 1, 2, 3, 4, 5, 6, 7, 8, 9, or 10 days after applying an alternating electric field is performed. In some aspects, administering a Notch1 inhibitor is performed simultaneously with applying an alternating electric field. In some aspects, administering a Notch1 inhibitor is performed within hours, days, or weeks of applying an alternating electric field.

In some aspects, nanoparticles can be used in the disclosed methods. For example, in some aspects, chelators, such as Notch1 inhibitors, can be carried by nanoparticles (e.g. encapsulated by nanoparticles) to provide specific chelation at the target site thus avoiding systemic influence of chelating calcium. In some aspects, the nanoparticles can be induced to release the Notch1 inhibitor in the presence of alternating electric fields. For example, in some aspects, alternating electric fields can cause the nanoparticle to burst thus releasing the Notch1 inhibitor. In some aspects, a nanoparticle can be a polymeric nanoparticle, liposome, micelle, metal nanoparticles. In some aspects, the nanoparticle comprises a target site-specific targeting moiety. In some aspects, the target site-specific targeting moiety can be a cancer cell-specific targeting moiety. In some aspects, the targeting moiety can direct, or target, the nanoparticle to a specific target site. The target site-specific targeting moiety can be a chemical, compound, peptide or nucleic acid. Examples of targeting moieties include, but are not limited to, molecules that recognize receptors on specific cell types.

### E. Method of Reducing Cancer Cell Growth

Disclosed are methods of reducing cancer cell growth comprising: applying an alternating electric field to a population of cells comprising one or more cancer cells; and contacting a Notch1 inhibitor to the population of cells. In some aspects, the population of cells is in vitro. In some aspects, the population of cells is in a subject. In some aspects, the alternating electric field is administered at a frequency for a period of time.

In some aspects, the Notch1 inhibitor is a γ Secretase Inhibitor. In some aspects, the γ Secretase Inhibitor is DAPT (GSI-IX), LY-411575, LY900009, RO4929097 (RG-4733), YO-01027 (Dibenzazepine), Crenigacestat (LY3039478), Semagacestat, BMS-906024, Avagacestat (BMS-708163), Sulindac sulfide, Dihydroergocristine mesylate (DHEC mesylate), γ-Secretase-IN-1, BMS 433796, L-685458, BMS 299897, MDL-28170, Nirogacestat, BT-GSI, E 2012, MK-0752, SPL-707, MRK-560, Itanapraced, JNJ-40418677, Z-Ile-Leu-aldehyde (Z-IL-CHO), or BT-GSI.

In some aspects, the Notch1 inhibitor is Jagged-1 (188-204) TFA, JI130, Procyanidin B2, 3,3′-di-O-gallate, Limantrafin (CB-103), Tangeretin (Tangeritin), Carvacrol, RBPJ Inhibitor-1 (RIN1), IMR-1, Psoralidin, BMS-906024 or Tarlatamab (AMG-757), Bruceine D, FLI-06, ZLDI-8, JI051, Rovalpituzumab, BMS-983970, BMS-986115, Brontictuzumab, IMR-1A, Tarextumab, Demcizumab, ASR-490, Navicixizumab, SAHM1, SAHM1 TFA, Enoticumab, FLI-06, SAHM1, NVS-ZP7-4, or JI051.

In some aspects, the Notch1 inhibitor is an anti-Notch1 antibody (mAb to DSL ligands, NRR antibodies, mAbs to Notch receptors, mAbs to Nicastrin, mAbs to ADAMS, mAbs to NICD, mAbs to MAM), a Notch1 decoy, Notch1 antagonist, siRNA, CRISPR/CAS, Y-secretase inhibitor, α-secretase inhibitor, acidification inhibitor, or a MAM stapled peptide.

In some aspects, the cancer cells can be, but are not limited to, a brain cancer cell, ovarian cancer cell, breast cancer cell, thyroid cancer cell, kidney cancer cell, lung cancer cell, digestive organ cancer cell, colon cancer cell, or endometrial cancer cell.

In some aspects, the Notch1 inhibitor reduces cancer cell growth. In some aspects, the Notch1 inhibitor reduces cancer cell proliferation. In some aspects, the Notch1 inhibitor reduces metastasis.

In some aspects, the alternating electric field is applied before, after, or simultaneously with administering the Notch1 inhibitor. In some aspects, the step of applying the alternating electric fields begins at least one hour before a Notch1 inhibitor is administered. In some aspects, when the Notch1 inhibitor is administered before applying the alternating electric fields, the cells are surprisingly sensitized to the alternating electric fields, thereby increasing the efficacy of the alternating electric fields. In some aspects, the step of applying the alternating electric fields begins at least 30 minutes before administering a Notch1 inhibitor. In some aspects, applying the alternating electric fields simultaneously can mean applying within 5, 10, 15, 20, 25, 30, 35, 40, 45, 50, 55, or 60 minutes before or after administering an Notch1 inhibitor. In some aspects, the alternating electric fields can be applied and the Notch1 inhibitor administered at least 1, 2, 3, 4, 5, 6, 7, 8, 9, 10, 11, 12, 13, 14, 15, 16, 17, 18, 19, 20, 21, 22, 23, or 24 hours from each other.

In some aspects, the Notch1 inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

In some aspects, the frequency and/or field strength of the alternating electric field can be any of those described herein and can be applied in any of the manners described herein. In some aspects, the frequency of the alternating electric field is between 50 kHz and 1 MHz. In some aspects, the frequency of the alternating electric field is about 150 or 250 kHz. In some aspects, the alternating electric field has a field strength of between 0.5 and 10 V/cm RMS. In some aspects, the alternating electric field has a field strength of about 0.9 V/cm RMS.

In some aspects, the methods of reducing cancer cell growth further comprise contacting the population of cells with a cancer therapeutic. In some aspects, a cancer therapeutic can be any known cancer therapeutic, such as, but not limited to, a chemotherapeutic agent or anti-inflammatory agent.

In some aspects, after applying an alternating electric field and prior to contacting the population of cells with a Notch1 inhibitor, detecting an increase in Notch1 expression in one or more cells of the population of cells. Thus, in some aspects, the method comprises only contacting the population of cells with a Notch1 inhibitor to those cells having an increase in Notch1 after applying an alternating electric field.

In some aspects, administering a Notch1 inhibitor is performed 1, 2, 3, 4, 5, 6, 7, 8, 9, or 10 days after applying an alternating electric field is performed. In some aspects, contacting the population of cells with a Notch1 inhibitor is performed simultaneously with applying an alternating electric field. In some aspects, contacting the population of cells with a Notch1 inhibitor is performed within hours, days, or weeks of applying an alternating electric field.

In some aspects, nanoparticles can be used in the disclosed methods. For example, in some aspects, chelators, such as Notch1 inhibitors, can be carried by nanoparticles (e.g. encapsulated by nanoparticles) to provide specific chelation in the population of cells or at the target site thus avoiding systemic influence of chelating calcium. In some aspects, the nanoparticles can be induced to release the Notch1 inhibitor in the presence of alternating electric fields. For example, in some aspects, alternating electric fields can cause the nanoparticle to burst thus releasing the Notch1 inhibitor. In some aspects, a nanoparticle can be a polymeric nanoparticle, liposome, micelle, metal nanoparticles. In some aspects, the nanoparticle comprises a target site-specific targeting moiety. In some aspects, the target site-specific targeting moiety can be a cancer cell-specific targeting moiety. In some aspects, the targeting moiety can direct, or target, the nanoparticle to a specific target site. The target site-specific targeting moiety can be a chemical, compound, peptide or nucleic acid. Examples of targeting moieties include, but are not limited to, molecules that recognize receptors on specific cell types.

### F. Method of Sensitizing Cancer Cells to an Alternating Electric Field

Disclosed are methods of sensitizing cancer cells to an alternating electric field comprising: applying an alternating electric field to a population of cells comprising one or more cancer cells; and contacting a Notch1 inhibitor to the population of cells. In some aspects, sensitizing the cancer cells to an alternating electric field enhances the therapeutic efficacy of the alternating electric field. In some aspects, the population of cells is in vitro. In some aspects, the population of cells is in a subject. In some aspects, the alternating electric field is administered at a frequency for a period of time.

In some aspects, the Notch1 inhibitor is a γ Secretase Inhibitor. In some aspects, the Y Secretase Inhibitor is DAPT (GSI-IX), LY-411575, LY900009, RO4929097 (RG-4733), YO-01027 (Dibenzazepine), Crenigacestat (LY3039478), Semagacestat, BMS-906024, Avagacestat (BMS-708163), Sulindac sulfide, Dihydroergocristine mesylate (DHEC mesylate), γ-Secretase-IN-1, BMS 433796, L-685458, BMS 299897, MDL-28170, Nirogacestat, BT-GSI, E 2012, MK-0752, SPL-707, MRK-560, Itanapraced, JNJ-40418677, Z-Ile-Leu-aldehyde (Z-IL-CHO), or BT-GSI.

In some aspects, the Notch1 inhibitor is Jagged-1 (188-204) TFA, JI130, Procyanidin B2, 3,3′-di-O-gallate, Limantrafin (CB-103), Tangeretin (Tangeritin), Carvacrol, RBPJ Inhibitor-1 (RIN1), IMR-1, Psoralidin, BMS-906024 or Tarlatamab (AMG-757), Bruceine D, FLI-06, ZLDI-8, JI051, Rovalpituzumab, BMS-983970, BMS-986115, Brontictuzumab, IMR-1A, Tarextumab, Demcizumab, ASR-490, Navicixizumab, SAHM1, SAHM1 TFA, Enoticumab, FLI-06, SAHM1, NVS-ZP7-4, or JI051.

In some aspects, the Notch1 inhibitor is an anti-Notch1 antibody (mAb to DSL ligands, NRR antibodies, mAbs to Notch receptors, mAbs to Nicastrin, mAbs to ADAMS, mAbs to NICD, mAbs to MAM), a Notch1 decoy, Notch1 antagonist, siRNA, CRISPR/CAS, Y-secretase inhibitor, α-secretase inhibitor, acidification inhibitor, or a MAM stapled peptide.

In some aspects, the cancer cells can be, but are not limited to, a brain cancer cell, ovarian cancer cell, breast cancer cell, thyroid cancer cell, kidney cancer cell, lung cancer cell, digestive organ cancer cell, colon cancer cell, or endometrial cancer cell.

In some aspects, the Notch1 inhibitor reduces cancer cell growth. In some aspects, the Notch1 inhibitor reduces cancer cell proliferation. In some aspects, the Notch1 inhibitor reduces metastasis.

In some aspects, the alternating electric field is applied before, after, or simultaneously with administering the Notch1 inhibitor. In some aspects, the step of applying the alternating electric fields begins at least one hour before a Notch1 inhibitor is administered. In some aspects, when the Notch1 inhibitor is administered before applying the alternating electric fields, the cells are surprisingly sensitized to the alternating electric fields, thereby increasing the efficacy of the alternating electric fields. In some aspects, the step of applying the alternating electric fields begins at least 30 minutes before administering a Notch1 inhibitor. In some aspects, applying the alternating electric fields simultaneously can mean applying within 5, 10, 15, 20, 25, 30, 35, 40, 45, 50, 55, or 60 minutes before or after administering an Notch1 inhibitor. In some aspects, the alternating electric fields can be applied and the Notch1 inhibitor administered at least 1, 2, 3, 4, 5, 6, 7, 8, 9, 10, 11, 12, 13, 14, 15, 16, 17, 18, 19, 20, 21, 22, 23, or 24 hours from each other.

In some aspects, the Notch1 inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

In some aspects, the frequency and/or field strength of the alternating electric field can be any of those described herein and can be applied in any of the manners described herein. In some aspects, the frequency of the alternating electric field is between 50 kHz and 1 MHz. In some aspects, the frequency of the alternating electric field is about 150 or 250 kHz. In some aspects, the alternating electric field has a field strength of between 0.5 and 10 V/cm RMS. In some aspects, the alternating electric field has a field strength of about 0.9 V/cm RMS.

In some aspects, the methods of sensitizing cancer cells to an alternating electric field further comprise contacting the population of cells with a cancer therapeutic. In some aspects, a cancer therapeutic can be any known cancer therapeutic, such as, but not limited to, a chemotherapeutic agent or anti-inflammatory agent.

In some aspects, after applying an alternating electric field and prior to contacting the population of cells with a Notch1 inhibitor, detecting an increase in Notch1 expression in one or more cells of the population of cells. Thus, in some aspects, the method comprises only contacting the population of cells with a Notch1 inhibitor to those cells having an increase in Notch1 after applying an alternating electric field.

In some aspects, administering a Notch1 inhibitor is performed 1, 2, 3, 4, 5, 6, 7, 8, 9, or 10 days after applying an alternating electric field is performed. In some aspects, contacting the population of cells with a Notch1 inhibitor is performed simultaneously with applying an alternating electric field. In some aspects, contacting the population of cells with a Notch1 inhibitor is performed within hours, days, or weeks of applying an alternating electric field.

In some aspects, nanoparticles can be used in the disclosed methods. For example, in some aspects, chelators, such as Notch1 inhibitors, can be carried by nanoparticles (e.g. encapsulated by nanoparticles) to provide specific chelation in the population of cells or at the target site thus avoiding systemic influence of chelating calcium. In some aspects, the nanoparticles can be induced to release the Notch1 inhibitor in the presence of alternating electric fields. For example, in some aspects, alternating electric fields can cause the nanoparticle to burst thus releasing the Notch1 inhibitor. In some aspects, a nanoparticle can be a polymeric nanoparticle, liposome, micelle, metal nanoparticles. In some aspects, the nanoparticle comprises a target site-specific targeting moiety. In some aspects, the target site-specific targeting moiety can be a cancer cell-specific targeting moiety. In some aspects, the targeting moiety can direct, or target, the nanoparticle to a specific target site. The target site-specific targeting moiety can be a chemical, compound, peptide or nucleic acid. Examples of targeting moieties include, but are not limited to, molecules that recognize receptors on specific cell types.

### G. Delivery of Compositions

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, tri-alkyl and aryl amines and substituted ethanolamines.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of Notch1 inhibitors and one or more materials for delivering alternating electric fields, such as the Optune® system. For example disclosed are kits comprising one or more of a Notch1 inhibitor and one or more materials for delivering alternating electric fields, such as the Optune® system. In some aspects, the kits can also include a cancer therapeutic.

### EXAMPLES

### A. Example 1

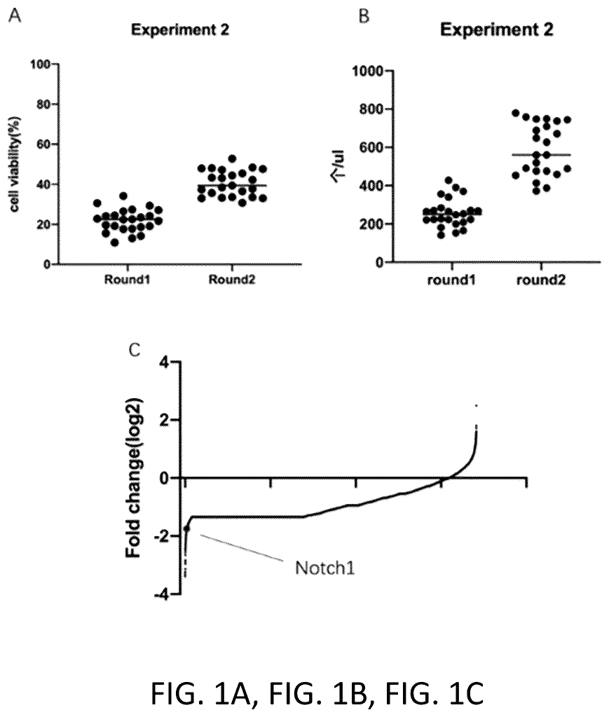

FIG. 1 shows an increased sensitivity to TTFields after disrupting the Notch1 gene.

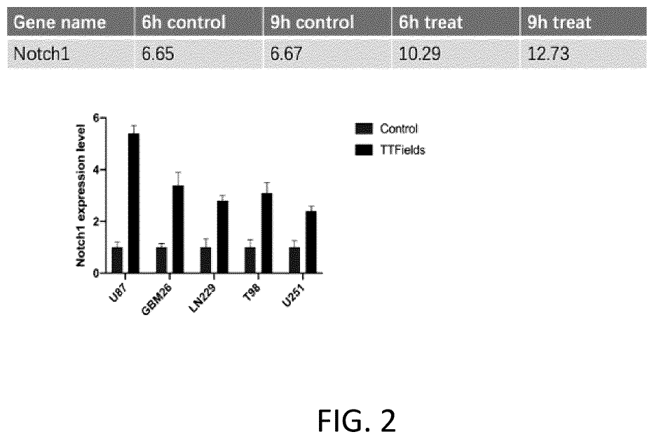

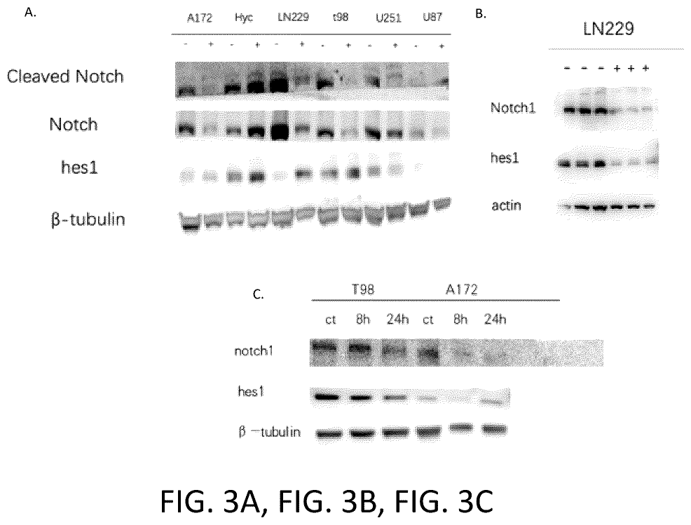

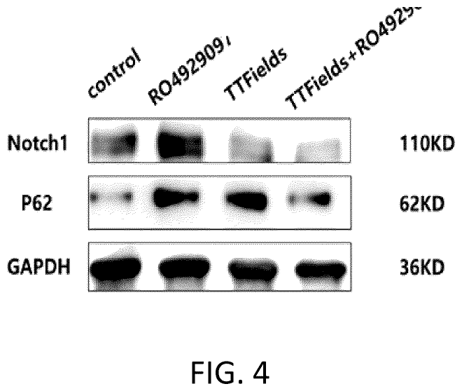

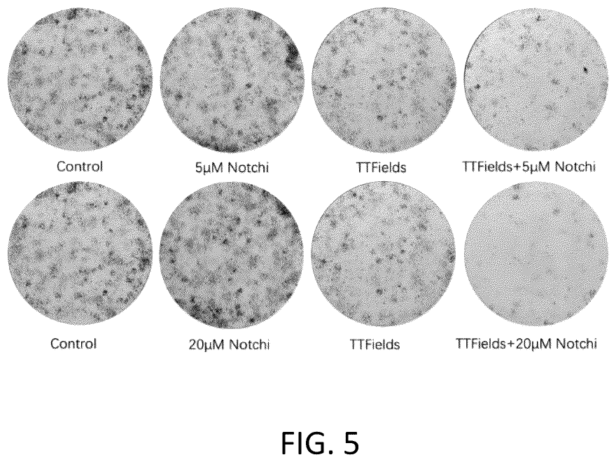

Treatment of cells with alternating electric fields results in an increase in Notch1 mRNA (FIG. 2) and degradation of Notch1 protein levels. (FIG. 3). However, the combination of a Notch1 inhibitor, RO4929097, with an alternating electric field sensitizes alternating electric field therapy (FIGS. 4 and 5).

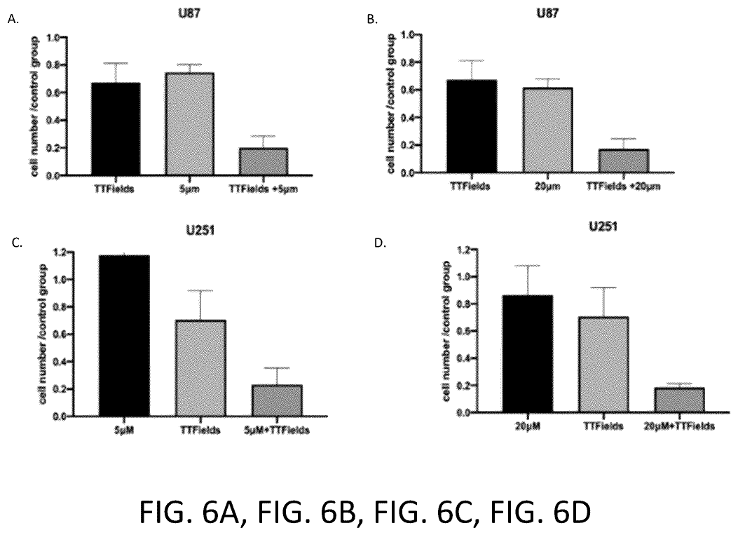

FIG. 6 shows that in glioblastoma cell lines U251 and U87 treatment with TTFields resulted in 60% cell viability in both cell lines; treatment with 5 μM Notch1 inhibitor resulted in 80% viability in U87 cells and no change in viability in U251 cells. The concomitant treatment of TTFields and 5 μM of Notch1 inhibitor resulted in 20% viability in both cell lines. Furthermore, treatment with 20 μM Notch1 inhibitor resulted in around 60% viability in both cell lines. The concomitant treatment of TTFields and 20 μM of Notch1 inhibitor resulted in 20% viability in both cell lines.

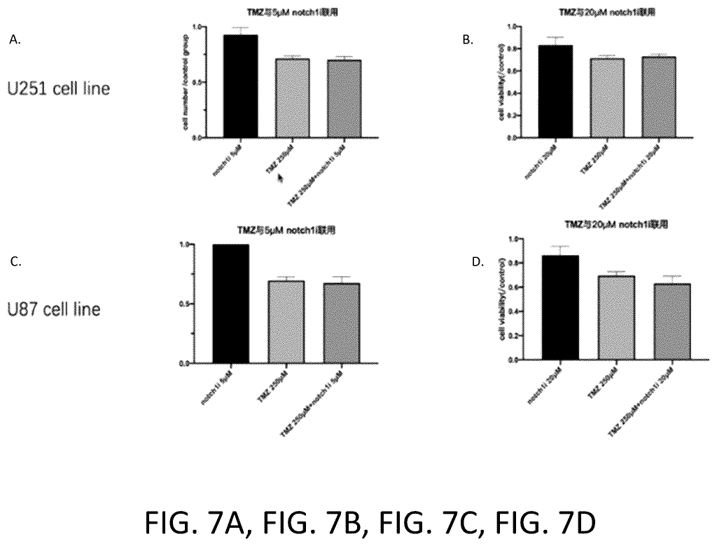

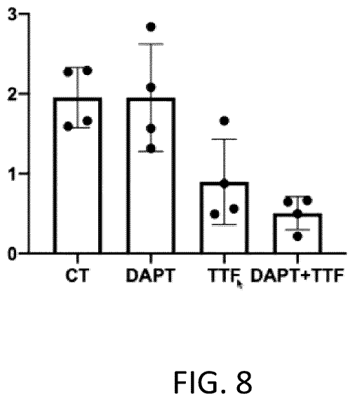

FIG. 7 shows that in glioblastoma cell lines U251 and U87 treatment with 250 μM of Temozolomide resulted in 75% cell viability in both cell lines and treatment with 5 μM Notch1 inhibitor resulted no change in viability in both cell lines. The concomitant treatment of 250 μM of Temozolomide and 5 μM of Notch1 inhibitor resulted in no change in viability in both cell lines. Furthermore, treatment with 20 μM Notch1 inhibitor resulted in around 80% viability in both cell lines. The concomitant treatment of 250 μM of Temozolomide and 20 μM of Notch1 inhibitor resulted in no change in viability in both cell lines.

### B. Example 2

### 1. Introduction

Glioblastoma (GB) is the most aggressive and prevalent primary brain tumor. Current standard of care involves surgery, followed by radiotherapy and chemotherapy. Even with optimal treatment, the prognosis for patients with this disease is poor, with a median survival of less than 2 year.

TTFields is an innovative and noninvasive therapeutic approach to cancer therapy. It represents a new anti-cancer mechanism and is deemed as the fourth modality in cancer treatment following chemotherapy, radiotherapy and immunotherapy. TTFields demonstrated safety and efficacy in patients with GB and has been approved by FDA for use in newly diagnosed GB and recurrent GB. Recently it was also approved by National Medical Products Administration (NMPA) in China for supratentorial newly diagnosed and recurrent GB. The use of electric fields for cancer therapy has received great enthusiasm.

A commonly accepted anticancer mechanism of TTFields is as follows: by exerting continuous low intensity, intermediate frequency, alternating electric fields on tumor site, TTFields disturb the orientation and movement of tubulins, thereby disrupting mitotic spindles and selectively killing rapidly dividing cancer cells. Other potential mechanisms have also been reported. It was argued that endoplasmic reticulum stress was induced by TTFields, which led to cellular stress and autophagy. TTFields were also demonstrated to repress the expression of crucial DNA repair genes such as BRCA1 and Fanconi anemia family genes, leading to enhanced sensitivity to ionizing radiation and possibly other DNA-damaging agents. It remains to be seen whether additional mechanisms underly TTFields sensitivity.

TTFields therapy has been proposed as a promising treatment for GB, and possibly other solid tumors. Yet, which genes may modify TTFields' effect on tumor, and whether certain genes strongly impact cellular survival during TTFields treatment remain unclear. Such knowledge is important for defining which subset of patients might benefit the most from TTFields therapy; it will also point to treatment combination that could enhance TTFields treatment outcome.

A genome-wide screen in TTFields-treated cancer cells may help answer the above question, possibly leading to a more comprehensive knowledge of TTFields' impact on cancer cells and resistance mechanism. In the field of glioblastoma research, glioma cell lines are naturally employed for conducting genome-wide CRISPR/Cas9 screens. However, such cell lines may not meet the need for TTFields in genome-wide screens. It is important to note TTFields research equipment, often in a 35 mm ceramic plate format, has a limited experimental capacity. A 200× coverage in a genome-wide CRISPR screen requires a cell quantity of 5×10⁷ cells, which would amount to more than 200 TTFields ceramic plates. Consequently, the undertaking of a genome-wide TTFields screen with glioma cells becomes unfeasible, as it demands the simultaneous operation of at least 20 inovitro systems.

To circumvent such limitations, in this study a genome-wide CRISPR screen was performed on an engineered cancer cell line, the Eu-Myc p19 Arf−/− lymphoma cell line. This cell line has proven to be a good model system for genome-wide CRISPR screens. First of all, such cells are non-adherent and small, with the size of only about 1/50 of glioma cells. Consequently, it can be seeded in high density (1.5×10⁶/ceramic plate), such that only about 30 TTFields ceramic plates are sufficient for a genome-wide CRISPR screen. In addition, this cell line has been successfully utilized to delineate gene-treatment interaction mechanisms in a number of studies. As a genetic engineered cell line with very few genetic lesions, it provides a relative clean genetic background for genome-wide screening. The cell line is also sensitive to external stress and has relative short cell doubling time, making it suitable for large scale screens.

Using the Eu-Myc p19 Arf−/− cell line, a systematic exploration was conducted of the anti-tumor mechanisms of TTFields using a genome-wide CRISPR/Cas9 library. An enrichment of genes associated with the Notch1 pathway was observed in the subset of depleted genes. Follow up studies in GB cell lines, organoids and TTFields-treated GB patient survival analysis implied that Notch1 represents a sensitization target for TTFields therapy.

### 2. Results

i. Genome-Wide CRISPR/Cas9 Screen Identifies Notch1 as a Potential Determinant of TTFields Sensitivity

Prior to this study, the Eu-Myc p19 Arf−/−cell line was used to perform genome-wide CRISPR screens on more than 40 anticancer drugs and achieved expected results. For example, in two screens with topoisomerase II poisons etoposide and doxorubicin, defects in topoisomerase II identified as a potent resistance mechanism, whereas defects in TDP2 and ZNF451, two genes capable of dissolving poisonous topoisomerase II-drug adducts, were identified as potent sensitization mechanisms (FIG. 15). Such data validated Eu-Myc p19 Arf−/− cell line as a suitable cellular background for conducting genome-wide CRISPR screens to study gene-treatment interaction.

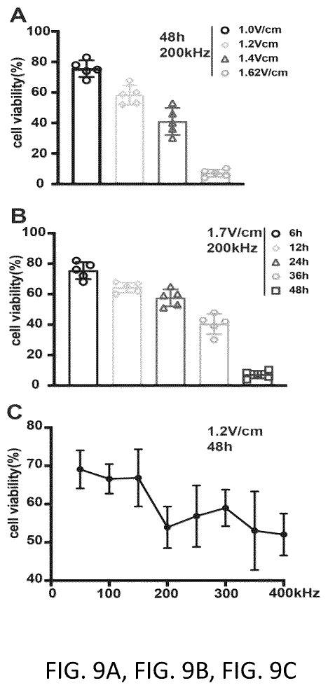

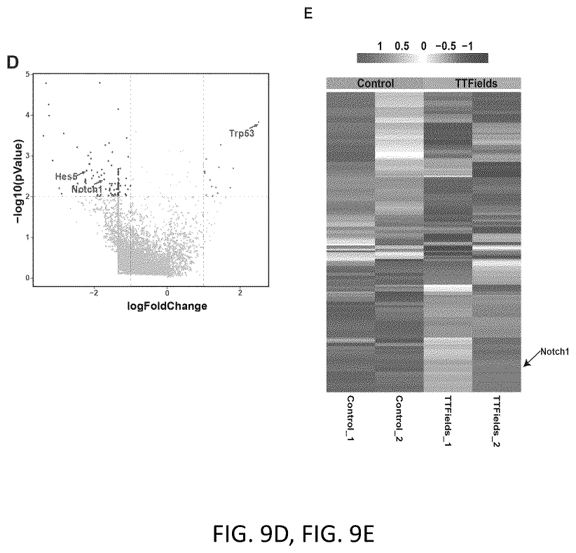

To determine the optimal parameters for TTFields screen, the cytotoxic effects of TTFields on Eu-Myc p19 Arf−/− cells were investigated across a spectrum of intensities, frequencies, and treatment durations. A gradual increase was observed in TTFields-induced cytotoxicity against Eu-Myc p19 Arf−/− cells with higher field intensities and longer treatment periods. Among the various frequencies tested, 200 kHz and 350 kHz emerged as particularly potent frequencies in eliciting death in Eu-Myc p19 Arf−/− cells (FIG. 9A-FIG. 9C). Given the primary focus on glioblastoma, the clinically relevant frequency of 200 kHz, commonly employed in glioma therapeutics, was used. The TTFields CRISPR screen was performed with an operating condition of 1.62V/cm, 200 kHz for a duration of 40 hours.

Finding potential sensitization methods for TTFields treatment was studied. Therefore, in the TTFields CRISPR screen results, genes whose deficiency results in less cell survival upon TTFields was focused on. Genes whose encoded proteins can be targets by clinical drugs were prioritized. In the TTFields CRISPR screen, Notch1 and Hes5 were both prominently enriched within the depleted gene set, indicating a potential enhancement in cellular susceptibility to TTFields treatment upon the ablation of Notch1 and Hes5 (FIG. 9D). A Notch1 inhibitor has been tested in clinical settings on several diseases and was recently approved for the treatment of Desmoid tumors. Therefore, it was decided to focus on Notch1 in this study. Notably, transcriptome analysis of TTFields-treated cells also revealed that Notch1 was amongst the most prominently upregulated genes upon TTFields treatment (FIG. 9E), which also prompted the prioritization of Notch1 in this study.

ii. Synergistic Sensitization to TTFields Therapy by Notch1 Inhibitors

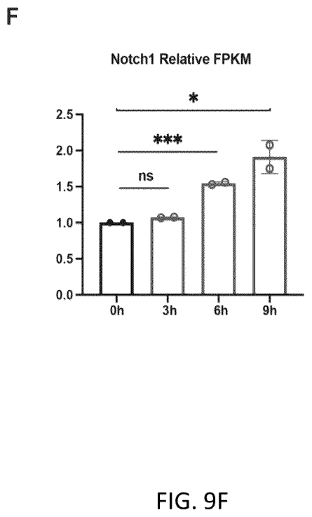

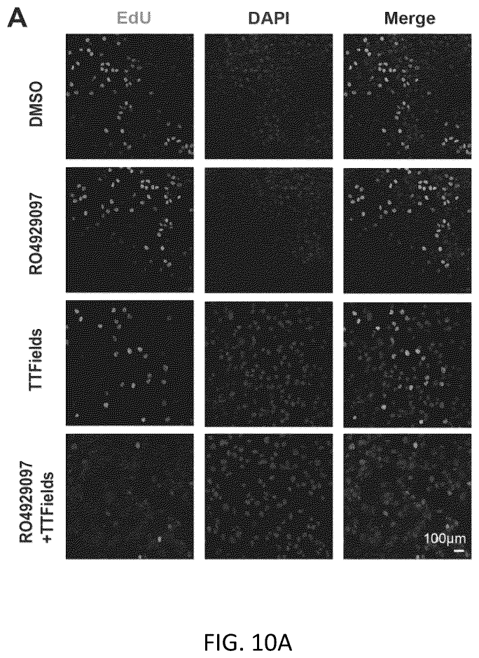

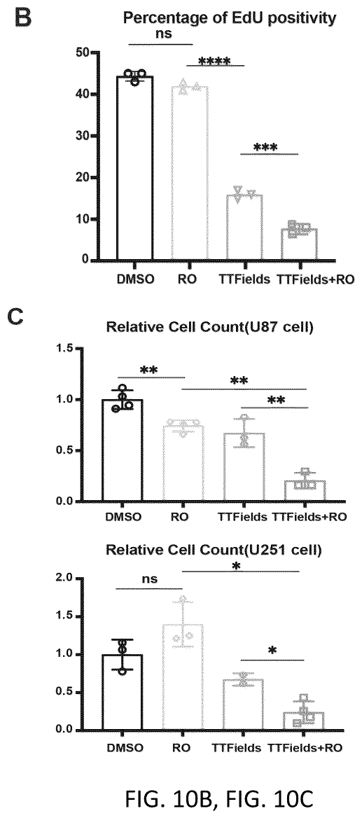

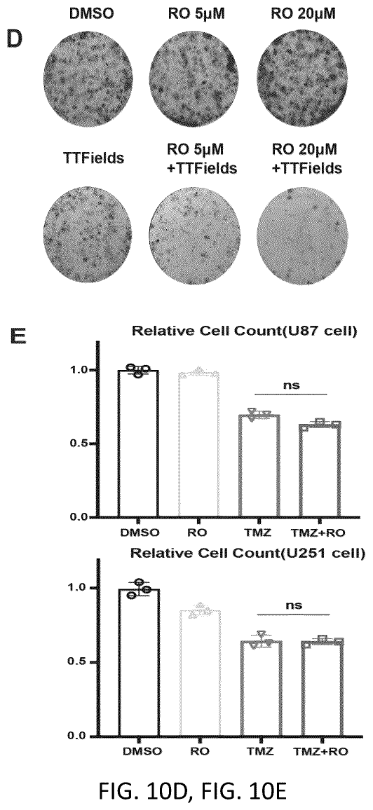

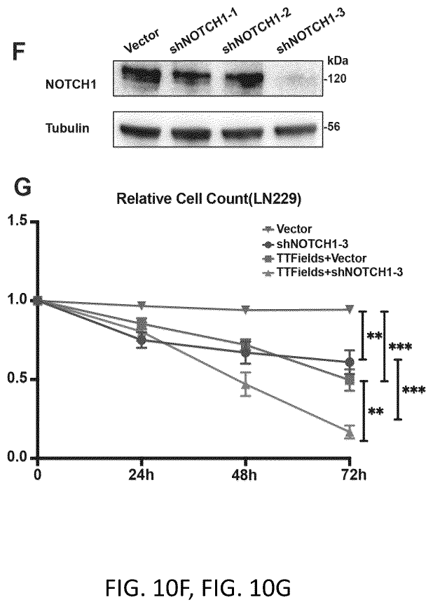

It was first analyzed whether Notch1 antagonization sensitized glioma cell lines to TTFields treatment. Validation assays were conducted across multiple glioma cell lines including U87, U251, and LN229. The Notch1 inhibitor RO4929097 was used to probe whether it synergizes with TTFields. In previous studies, RO4929097 has been tested in recurrent GB patients with favorable safety profiles. In the validation assays, experimental cohorts were divided into four groups: control (DMSO), TTFields treatment alone, Notch1 inhibitor RO4929097, and TTFields plus RO4929097. EdU incorporation assays showed minimal cytotoxicity in the RO4929097 group. Upon combining with TTFields, a noticeable enhancement in therapeutic sensitization became evident (FIGS. 10A and B). This observation was supported by cell counting assays conducted over a three-day period (FIG. 10C). Furthermore, long-term proliferative potential of treated cells, as evidenced by clonogenic assays, further confirmed the synergy between Notch1 inhibitor and TTFields on glioma cells (FIG. 10D). In contrast, RO4929097 did not sensitize glioma cells to temozolomide, a chemotherapeutic drug commonly used in glioma treatment (FIG. 10E).

It was further determined whether Notch1 knockdown could sensitize GB cell lines to TTFields treatment. Notch1 knockdown caused rapid cell death in U87 and A172 cells, rendering them unsuitable for prolonged combination experiments. LN229 cells were able to tolerate Notch1 knockdown and were used in further studies. Remarkably, upon Notch1 gene ablation in LN229 cells, a marked improvement in sensitivity to TTFields therapy was observed (FIGS. 10F and G).

iii. TTFields Induce Rapid Degradation of Notch1 via Lysosome.

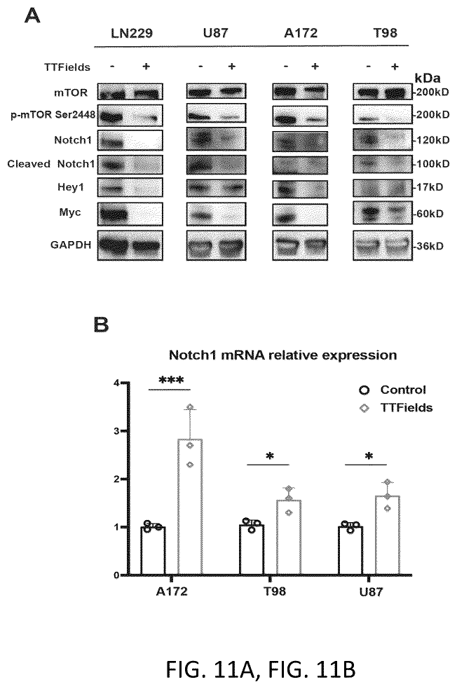

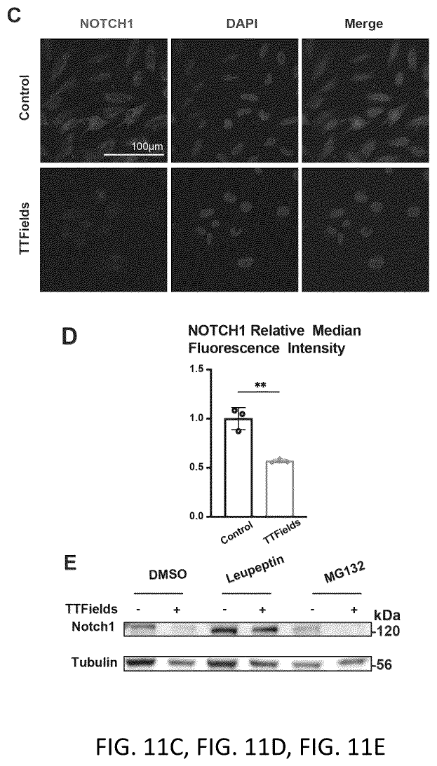

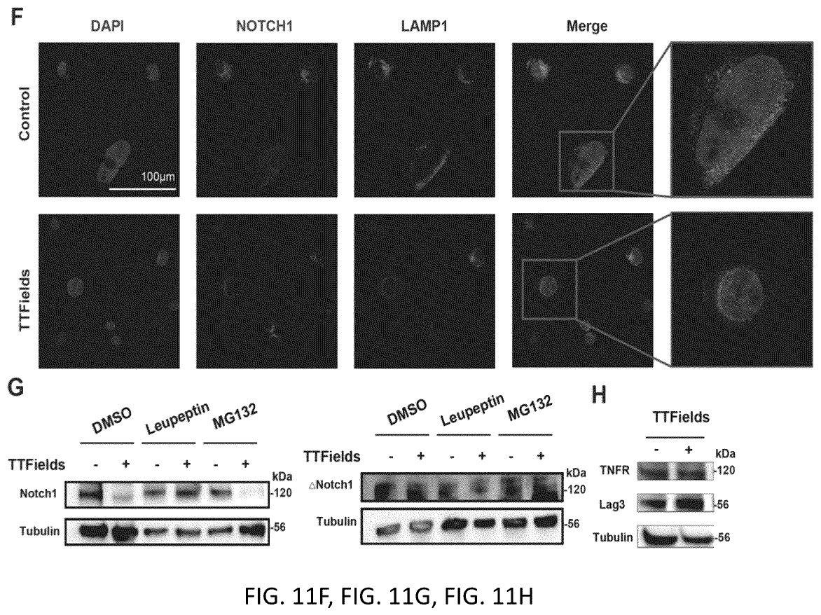

The status of Notch1 during TTFields treatment was analyzed. Western blot of cell lysates collected after exposure to TTFields revealed a reduction of mTOR p-S2448, which was consistent with previous literatures that TTFields inhibits mTOR activity. Interestingly, a substantial decrease in Notch1 protein levels, together with a decline in the intracellular cleaved form of Notch1, was also observed (FIG. 11A). Furthermore, downstream genes of the Notch1 pathway, including Hey1, Hes1 and Myc, also displayed marked downregulation upon TTFields treatment. RT-qPCR analyses revealed a contradictory trend: despite decreased protein levels of Notch1, its mRNA level showed an increase following TTFields treatment (FIG. 11B). This phenomenon indicates a cellular response mechanism wherein tumor cells, Notch1 activity is required for cellular survival upon TTFields treatment; under the influence of electric fields, Notch1 protein undergoes rapid degradation. To mitigate this destabilization, cells attempt compensation by upregulating Notch1 mRNA levels as a form of “self-rescue”. Disrupting this attempt via Notch1 inhibitors may hold promise in rendering cells more susceptible to TTFields therapy.

Reduction of Notch1 level by TTFields was also confirmed by immunofluorescence staining (FIGS. 11C and D). The intracellular protein degradation mechanism primarily involves proteasomal and lysosomal degradation pathways. To further elucidate the mechanism of TTFields-mediated degradation of Notch1 protein, lysosomal inhibitor Leupeptin and proteasomal inhibitor MG132 were employed (FIG. 11E). It was found that inhibition of the lysosomal degradation pathway, but not the proteasome pathway, blocked TTFields-induced degradation of Notch1 protein. Immunofluorescent staining also revealed that upon TTFields treatment, Notch1 colocalized with lysosome marker LAMP1 (FIG. 11F). Lysosomal degradation of proteins, particularly those located on cell membrane such as Notch1, often involves cell membrane. Full-length Notch1 or intracellular cleaved Notch1 was ectopically expressed in U87 cells and it was found that TTFields could degrade full-length Notch1 protein but not the cleaved Notch1 protein (FIG. 11G). This indicates that TTFields-induced lysosomal degradation of Notch1 can involve Notch1's membrane localization. Western blot analysis of several other plasma membrane proteins including TNFR and LAG3 indicated that TTFields treatment did not cause degradation of membrane proteins indiscriminately (FIG. 11H).

iv. TTFields-Mediated Degradation of Notch1 Induces Differentiation of Cancer Stem Cells

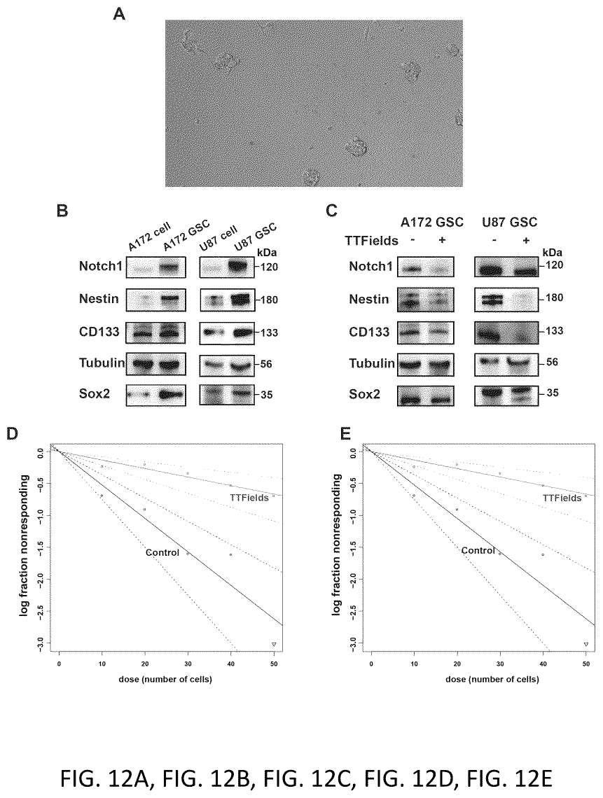

Many studies have underscored the pivotal role of the Notch1 gene in maintaining the stemness of glioma cells. Therefore, it was postulated that TTFields, with its degrading effect on Notch1, might reduce the stemness of tumor-initiating cells. Tumor-initiating cells were induced from U87 and A172 cells using tumor stem cell culture medium. Western blot results indicated successful induction, with significant upregulation of proteins such as Notch1, Nestin, Sox2, and CD133 (FIG. 12B). Upon TTFields treatment of tumor-initiating cells, there was a noticeable reduction in Notch1 protein levels, as well as in the protein levels of Nestin, Sox2, CD133 and other stemness marker genes (FIG. 12C). Additionally, results from the limiting dilution sphere-forming assay demonstrated a significant decrease in the sphere-forming ability of tumor-initiating cells in TTFields-treated group (FIGS. 12D and E).

v. Glioblastoma Organoid Model Validates Notch1 as a Sensitization Target for TTFields Treatment

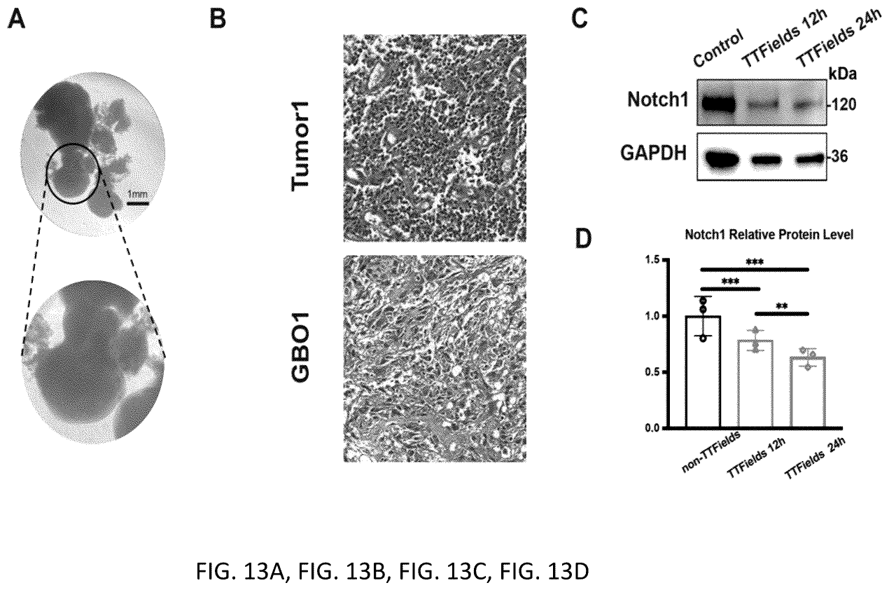

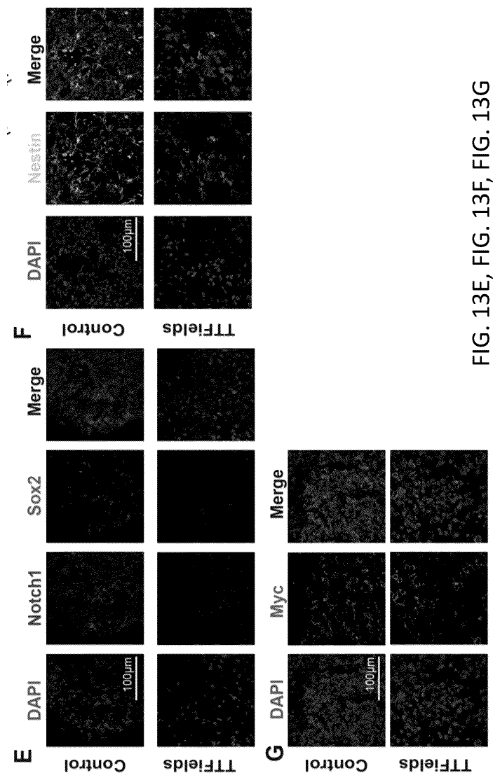

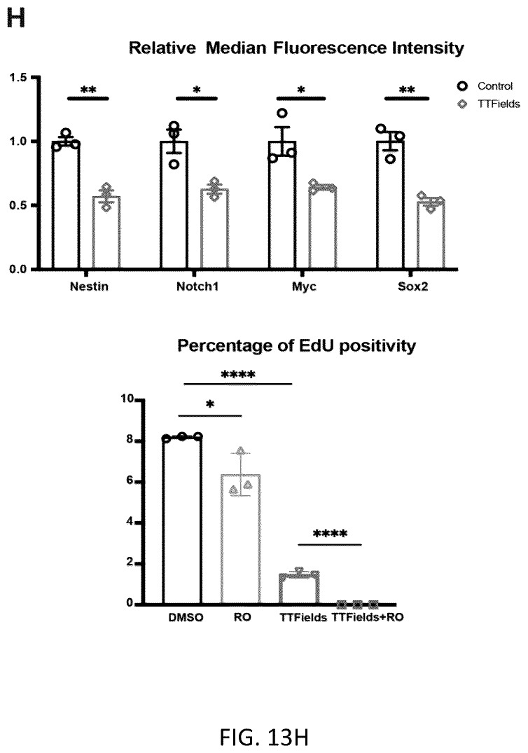

To further confirm the effects of TTFields on glioblastoma and the synergistic effect of Notch1 inhibitor with TTFields, patient-derived glioblastoma organoids (GBOs) were constructed. Under bright-field microscopy, the organoids exhibited a rounded morphology with a dense center and smooth periphery (FIG. 13A). Subsequently, the GBOs were characterized. Histopathological analysis with H&E staining revealed that GBOs closely resembled the tumor morphology of the parental tumor tissue, displaying complex cellular compositions including various tumor cell morphologies and neuronal components (FIG. 13B). Genome sequencing also indicated that GBO faithfully represented the parental tumor. Next, GBOs were subjected to TTFields treatment. It was found that TTFields could similarly reduce Notch1 protein level in GBOs. Moreover, with increasing treatment duration, the reduction Notch1 protein level became more pronounced (FIGS. 13C and D). Immunofluorescence results demonstrated that in GBOs, TTFields could reduce the level of Notch1 as well as other genes such as Sox2, Nestin, and Myc, further demonstrating TTFields' ability to reduce the stemness of tumor cells (FIG. 13E to H). Subsequently, whether the Notch1 inhibitor RO4929097 could exhibit synergistic effects with TTFields in GBOs was addressed. Similarly, the GBOs were divided into four groups: control (DMSO), TTFields treatment group, Notch1 inhibitor RO4929097 alone group and combination group. Results from Edu staining indicated that when used alone, the Notch1 inhibitor showed no apparent cytotoxicity, whereas when combined with TTFields, a clear synergistic effect was observed (FIG. 13I).

vi. Clinical Validation of Notch1 as a Determinant of TTFields Treatment Outcome

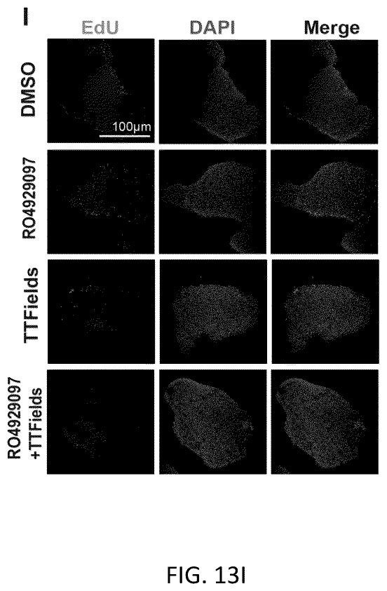

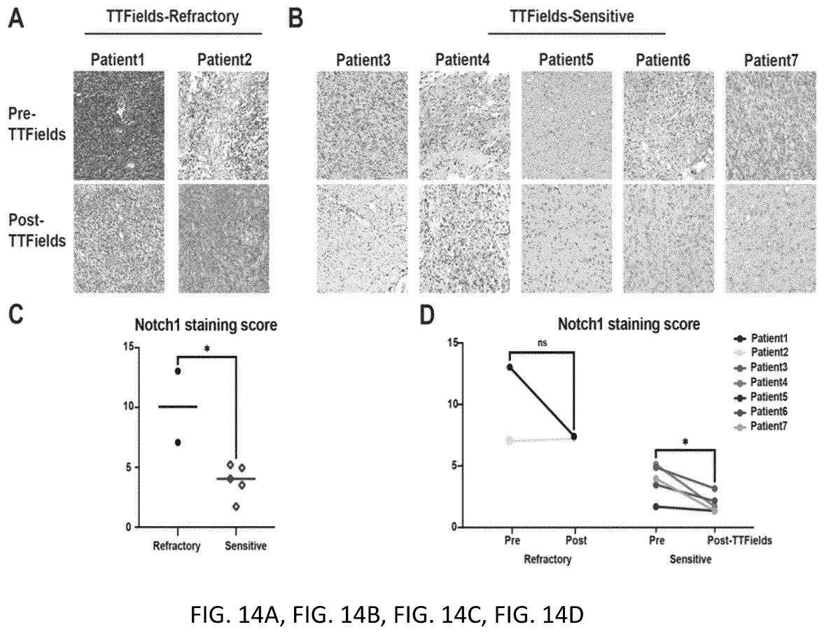

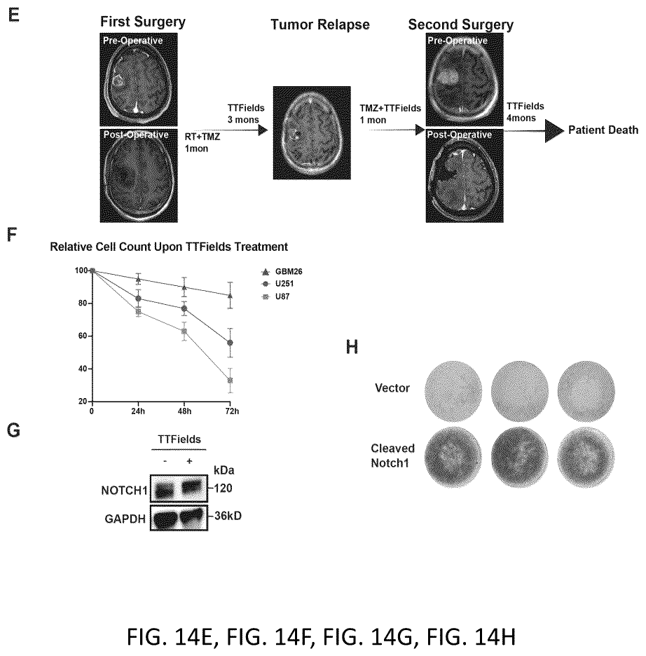

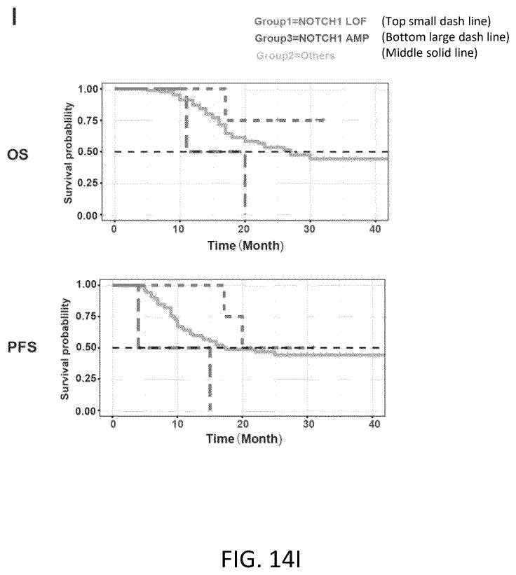

Between 2017 and 2022, over 300 primary glioblastoma patients were treated at Huashan with TTFields therapy. Among them, over 200 were excluded due to incomplete follow-up data or treatment duration of less than three months. A cohort of 100 patients had complete follow-up data, with 73 of which having tumor sequencing data, and were thus included in the analysis of Notch1 pathway alterations and sensitivity to TTFields therapy. Tumor resection surgery was performed on seven TTFields-treated GB patients after tumor recurrence. All patients underwent extensive tumor resection and received Stupp regimen therapy. Patients with progression-free survival (PFS)<8 months were considered TTFields-refractory, while those with PFS>10 months were deemed sensitive to TTFields therapy. Using immunohistochemistry staining, Notch1 protein level was analyzed in these seven GBs before and after TTFields treatment. The result showed that in TTFields-naïve tumor samples, GBs in the TTFields-refractory group had significantly higher Notch1 levels than those in the TTFields-sensitive group (FIG. 14A to C). Moreover, GBs sensitive to TTFields showed a significant decrease in Notch1 immunohistochemical score after TTFields treatment. In one GB refractory to TTFields (PFS=4 month), TTFields failed to reduce Notch1 level. In the other TTFields-refractory GB (PFS=7 month), TTFields caused reduction in Notch1 level; however, the Notch1 level post treatment was still higher than Notch1 levels in the pretreatment samples from the TTFields-sensitive group (FIG. 14D). These data indicate that a difference in Notch1 level can impact TTFields sensitivity, which is consistent with the finding from the cellular studies.

Here, a typical case of a GB refractory to TTFields therapy is presented (Patient 2). The patient underwent the first surgery in January 2021, with postoperative pathology confirming IDH1-negative glioblastoma. After a month of recovery, the patient commenced radiotherapy and concurrent temozolomide chemotherapy, followed by TTFields therapy and adjuvant chemotherapy with temozolomide. However, tumor recurrence was evident just three months later. A second tumor resection surgery was performed, and tumor cells were extracted during the procedure. Postoperative MRI revealed complete tumor resection. After a month of recovery, the patient received TTFields therapy again, but the tumor recurred four months later, ultimately leading to the patient's demise (FIG. 14E). Primary cells extracted during the second surgery (GB26) were treated with TTFields in vitro for 72 hours, revealing significant resistance to TTFields therapy compared to U87 and A172 cell lines (FIG. 14F). Notably, in such TTField-refractory tumor cells, TTFields failed to induce degradation of Notch1 protein (FIG. 14G). Ectopic expression of cleaved Notch was used in a GB A172 cell line to model a situation in which cells can retain Notch level despite TTFields treatment (FIG. 14H). Results of this experiment clearly demonstrated that when cells were equipped with Notch level that was not affected by TTFields, they gained marked resistance to TTFields.

The correlation between Notch genetic status and prognosis was analyzed in 73 TTFields-treated GBs with genetic data. Based on the presence or absence of Notch1 pathway gene alterations, patients were categorized into Notch1 amplification group, Notch1 inactivation group, and others. The results showed that patients in the Notch1 amplification group (n=2) had the worst prognosis, while patients in the Notch1 pathway gene inactivation group (n=5) had a better prognosis compared to the group with no alterations (FIG. 14I).

These results support the experimental findings at the clinical level, indicating that elevated levels of the Notch1 gene may confer resistance to TTFields therapy. On the other hand, Notch1 inhibition can potentially sensitize GB cells to TTFields treatment. Based on these findings, GB patients can benefit from combination therapy with the Notch1 inhibitor RO4929097, potentially enhancing sensitivity to TTFields therapy and improving patient survival.

### 1. Discussion

As the understanding of the anti-tumor mechanisms of TTFields continues to evolve, the complexity of TTFields' anti-tumor mechanisms may exceed the initial expectations. This study is the first to employ a genome-wide approach, utilizing CRISPR/Cas9 screening, to explore the anti-tumor mechanisms of TTFields. Through this methodology, the pivotal role of Notch1 was identified in mediating resistance to TTFields therapy.

Upon exposure to TTFields, Notch1 was degraded. Cells exhibit upregulation of Notch1 transcription, presumably as an attempt to mitigate the loss of Notch1. Inhibition of Notch1 renders cells more susceptible to TTFields therapy. RO4929097, a small-molecule Notch1 inhibitor, exerts its effects by inhibiting γ-secretase and blocking downstream Notch1 signaling. Notably, RO4929097 has demonstrated promising outcomes in phase I and II clinical trials for glioblastoma, exhibiting favorable blood-brain barrier penetration and the ability to reduce expression of Notch1 downstream genes in tumor cells, with a favorable safety profile. Hence, RO4929097 holds promise as a potential sensitizer for TTFields therapy.

The results elucidated that the degradation of Notch1 protein relies on the lysosomal pathway, which is consistent with the biological effects of TTFields. TTFields have been shown to activate lysosomes, as evidenced by an increase in lysosomal abundance observed via electron microscopy following TTFields treatment. The lysosomal degradation of Notch1 protein by TTFields appears to be membrane-dependent. Potential mechanisms may involve the activation of certain genes under the influence of the electric field, leading to interactions with Notch1 protein and guiding its membrane trafficking and lysosomal degradation.

Numerous studies have underscored the crucial role of Notch1 gene in maintaining the stemness of tumor cells. This study unveils the capability of TTFields to diminish the stemness of tumor cells through the degradation of Notch1 protein. In a study investigating the effects of TTFields treatment on normal brain tissue organoids, downregulation of genes such as Sox2 and Tubulinβ-III was observed. Given that Sox2 was a transcription target of Notch1, the study uncovers the underlying mechanism behind this phenomenon, indicating that TTFields can achieve such effects by downregulating Notch1 protein. Given the pivotal role of tumor stem cells in tumor recurrence, this finding holds significant clinical implications. Unlike the cytotoxic side effects associated with radiotherapy, TTFields are generally believed to spare normal neuronal cells from cytotoxicity. Considering these distinctive features, the comprehensive coverage of tumor cavities and peritumoral regions (SVZ zone) with high tumor stem cell content in the application of TTFields is advocated for, thereby mitigating the stemness of tumor cells within the tumor and ultimately improving patient prognosis.

The clinical data corroborate the findings of the experimental study indicating that GBs with high Notch1 expression levels can exhibit reduced sensitivity to TTFields therapy. In TTFields-sensitive GBs, which generally express lower level of Notch1, TTFields treatment led to further decrease in Notch1 protein level. Such a reduction of Notch1 was not observed in GBs refractory to TTFields, which also exhibited higher Notch1 levels prior to TTFields treatment. However, the factors influencing patient prognosis are multifaceted, encompassing tumor malignancy, sensitivity to radiotherapy and chemotherapy, as well as age, among others. The prognostic significance of Notch1 gene expression in glioblastoma remains contentious, with research indicating potential prognostic implications primarily in the classical subtype of glioblastoma, while its significance in other subtypes remains inconclusive. In this study, all patients included were pathologically confirmed to have IDH1-negative, WHO grade IV glioblastoma, with complete tumor resection and receipt of full Stupp protocol treatment, ensuring maximal consistency across cohorts.

### 3. Methods

i. Cell Culture and Growth Conditions

HEK-293T, U87, A172, LN229, and T98 cells were obtained through purchase from the Shanghai Cell Bank of the Chinese Academy of Sciences (Shanghai, China). Cells were cultured in DMEM, 10% FBS medium at 37° C., 5% CO2. Eu-Myc p19Arf−/−mouse B cell lymphoma cells were cultured in B cell culture medium.

ii. Isolation of Culture of GICs

After dissociation of U87 and A172 cells, they were washed twice with PBS and then cultured in serum-free DMEM/F12 supplemented with 20 ng/ml basic fibroblast growth factor (bFGF; Life Technologies), 20 ng/ml epidermal growth factor (EGF; Life Technologies), and 2% B27 (Life Technologies). The cell culture medium was changed every three days, and after 1-2 weeks, the cells began to exhibit spherical aggregation. The stem-like properties of the cells were confirmed through Western Blot analysis.

iii. GBO Generation and Verification

The GBOs were generated based on known methods. Briefly, fresh frozen pathology-confirmed tumor samples, diagnosed as glioblastoma, were stored in Hibernate medium (Thermo Fisher Scientific) at 4° C. and promptly transferred from the operating room to the laboratory. Tumor-rich regions were identified under a microscope, and necrotic areas were excluded. The tumor was then sectioned into 1 mm3 pieces and placed in GBO medium consisting of 50% DMEM/F12 (Thermo Fisher Scientific), 50% Neurobasal (Thermo Fisher Scientific), 1× GlutaMax (Thermo Fisher Scientific), 1×NEAAs (Thermo Fisher Scientific), 1× PenStrep (Thermo Fisher Scientific), 1× N2 supplement (Thermo Fisher Scientific), 1× B27 w/o vitamin A supplement (Thermo Fisher Scientific), 1× 2-mercaptoethanol (Thermo Fisher Scientific), and 2.5 mg/ml human insulin (Sigma) within low-adhesion 12-well plates. The cultures were maintained at 37° C. with agitation at 300 rpm. After approximately one week, successful establishment was confirmed by observing obvious proliferation of GBOs, characterized by smooth, round borders. Validation was performed using techniques such as HE staining and genetic sequencing.

iv. Glioblastoma Primary Cell Culture

GB26 was isolated from tumor tissue obtained from a patient insensitive to TTFields treatment. Following confirmation by frozen pathology, indicating the tissue's glioblastoma status, the tissue was preserved in 4° C. DMEM and swiftly transferred to the laboratory. The tumor surface was rinsed with HBSS to remove residual blood. Tumor-rich areas were identified, and the tumor was then dissected into small fragments using scissors. These fragments were subjected to digestion using tumor dissociation kit (jingneng) at 37° C. for approximately 30 minutes. The resulting dissociated cell mixture was filtered through a 70 μm filter membrane to remove debris and underwent red blood cell removal. The remaining cells were seeded onto culture dishes and cultured in DMEM supplemented with 10% FBS. The culture medium was refreshed every 3 days. Upon observing significant cell proliferation, a portion of the cells was subjected to GFAP staining. Combined with the final pathology results, the successful establishment of GB26 primary glioblastoma cells was confirmed.

v. In Vitro Experiments

For short-term experiments, such as detecting the degradation process of glioblastoma cells under TTFields, U87, U251, LN229, and T98 cell suspensions (450 μL, 100,000 cells per well) were evenly spread on 24 mm cell culture slides. After overnight incubation, the cells were transferred to in vitro dishes and supplemented with 2 mL fresh culture medium. The treatment frequency was 200 kHz, with a treatment field strength of 1.62 V/cm RMS, for a duration of 24 hours. For long-term combination experiments, involving the combined use of RO4929097 or Notch1 gene knockdown with TTFields, cells (450 μL, 40,000 cells per well) were treated with a field strength of 1.19 V/cm RMS and a frequency of 200 kHz for 72 hours. The concentration of RO4929097 drug was varied. For organoid experiments, 3-5 GBOs were added to each well, and fresh GBO medium was replaced. The treatment field strength was 1.19 V/cm RMS, with a frequency of 200 kHz, for treatment durations of 24 and 48 hours.

vi. CRISPR-Cas9 Knockout Screen

The construction of the Cas9-expressing Eu-Myc p19Arf−/−stable cell line and CRISPR dropout/enrichment screens were performed. Cells were seeded at a density of 7.5×10⁵/ml, with a cell volume of 1.5 ml per dish. The cells were subjected to an electric field strength of 1.62V/m. To prevent excessive evaporation of the culture medium, the dishes were tightly sealed using Parafilm. After a 24-hour treatment with the Inovitro system, all cells were harvested, underwent centrifugation, and were then resuspended in fresh culture medium. Sealed once more with Parafilm, the cells were exposed to additional rounds of electric field treatment. After being exposed to the electric field for approximately 40 hours, the cell viability was reduced to around 20%-30%. Upon the cells reaching 100% viability again, they were subjected to the same conditions for a second round of treatment, repeating the aforementioned process. Cells were collected for genome extraction, and bioinformatic analysis was conducted using the MAGeCK (Model-based Analysis of Genome-wide CRISPR-Cas9 Knockout) algorithm (Li et al., 2014).

vii. Western Blot Analysis

Protein levels were evaluated through Western Blot analysis, following the established protocol. The antibodies employed in the study are listed below. Rabbit notch1 (Abcam, ab52627), Rabbit Cleaved Notch1 (CST, 4147S), Mouse anti-GAPDH (Abclonal, A19056), Rabbit anti-Tubulin (Abclonal, AC015), Rabbit anti-Hes1 (ab108937), rabbit anti-Hes5 (Abclonal A16237), rabbit anti-Hey1 (Abclonal A16110), rabbit anti-Myc (Selleck, A5011), anti-rabbit CD133 (Abcam, ab19898), anti-mouse Sox2 (Abcam, ab79351), anti-Rabbit Nestin (Abcam), and Anti-mouse lamp1.

viii. Colony Formation Assay

Following treatment with TTFields and corresponding conditions, 500-1000 cells were selected from each group and seeded in 6-well dishes. The cells were cultured at 37 degrees Celsius with 5% CO2 for a period of 7-10 days, with medium replacement every 3 days. After 10 days, cells were harvested, subjected to two PBS washes, fixed and stained using formaldehyde and crystal violet, and subsequently photographed for analysis.

ix. Statistics Analysis

All values are expressed as mean±standard error (mean). Statistical significance was evaluated by two-way repeated ANOVA analysis for cell and tumor growth curves or two-tailed Student's t-tests for all others, using GraphPad Prism 8. The statistical significance levels were set at *p<0.05, **p<0.01 and ***p<0.001. ANOVA was used to determine the statistical difference between tumor and paired normal samples acquired from GEPIA interactive web server. The cutoffs of Log 2FC and q-value are set at 1 and 0.05, respectively.

Those skilled in the art will recognize, or be able to ascertain using no more than routine experimentation, many equivalents to the specific embodiments of the method and compositions described herein. Such equivalents are intended to be encompassed by the following claims.

### EMBODIMENTS

Embodiment 1. A method of treating a subject in need thereof comprising applying an alternating electric field to a target site of the subject in need thereof; and administering a Notch1 inhibitor or Hes5 inhibitor to the subject in need thereof.
Embodiment 2. The method of embodiment 1, wherein the alternating electric field is administered at a frequency for a period of time.
Embodiment 3. The method of any of the preceding embodiments, wherein the Notch1 inhibitor is a γ Secretase Inhibitor.
Embodiment 4. The method of embodiment 3, wherein the γ Secretase Inhibitor is DAPT (GSI-IX), LY-411575, LY900009, RO4929097 (RG-4733), YO-01027 (Dibenzazepine), Crenigacestat (LY3039478), Semagacestat, BMS-906024, Avagacestat (BMS-708163), Sulindac sulfide, Dihydroergocristine mesylate (DHEC mesylate), γ-Secretase-IN-1, BMS 433796, L-685458, BMS 299897, MDL-28170, Nirogacestat, BT-GSI, E 2012, MK-0752, SPL-707, MRK-560, Itanapraced, JNJ-40418677, Z-Ile-Leu-aldehyde (Z-IL-CHO), or BT-GSI.
Embodiment 5. The method of embodiments 1-2, wherein the Notch1 inhibitor is, Jagged-1 (188-204) TFA, JI130, Procyanidin B2, 3,3′-di-O-gallate, Limantrafin (CB-103), Tangeretin (Tangeritin), Carvacrol, RBPJ Inhibitor-1 (RIN1), IMR-1, Psoralidin, BMS-906024 or Tarlatamab (AMG-757), Bruceine D, FLI-06, ZLDI-8, JI051, Rovalpituzumab, BMS-983970, BMS-986115, Brontictuzumab, IMR-1A, Tarextumab, Demcizumab, ASR-490, Navicixizumab, SAHM1, SAHM1 TFA, Enoticumab, FLI-06, SAHM1, NVS-ZP7-4, or JI051.
Embodiment 6. The method of embodiments 1-2, wherein the Notch1 inhibitor is an anti-Notch1 antibody (mAb to DSL ligands, NRR antibodies, mAbs to Notch receptors, mAbs to Nicastrin, mAbs to ADAMS, mAbs to NICD, mAbs to MAM), a Notch1 decoy, Notch1 antagonist, siRNA, CRISPR/CAS, Y-secretase inhibitor, α-secretase inhibitor, acidification inhibitors, MAM stapled peptides.
Embodiment 7. The method of any one of embodiments 1-6, wherein the subject has cancer.
Embodiment 8. The method of embodiment 7, wherein the cancer is glioblastoma, ovarian cancer, non-small cell lung cancer, breast cancer (e.g. triple-negative breast cancer), papillary Thyroid cancer, clear cell renal cell carcinoma, hepatocellular cancer, pleural mesothelioma, pancreatic cancer, lung cancer, gastric cancer, colon cancer or endometrial carcinoma.
Embodiment 9. The method of any one of embodiments 1-8, wherein the Notch1 inhibitor or Hes5 inhibitor reduces cancer cell growth.
Embodiment 10. A method of preventing cancer recurrence in a subject having cancer comprising applying an alternating electric field to a target site of the subject in need thereof; and administering a Notch1 inhibitor or Hes5 inhibitor to the subject in need thereof.
Embodiment 11. A method of reducing cancer cell growth comprising applying an alternating electric field to a population of cells comprising one or more cancer cells; and contacting a Notch1 inhibitor or Hes5 inhibitor to the population of cells.
Embodiment 12. A method of sensitizing cancer cells to an alternating electric field comprising applying an alternating electric field to a population of cells comprising one or more cancer cells; and contacting a Notch1 inhibitor or Hes5 inhibitor to the population of cells.
Embodiment 13. The method of embodiments 10-12, wherein the alternating electric field is administered at a frequency for a period of time.
Embodiment 14. The method of any one of embodiments 10-13, wherein the Notch1 inhibitor is, Jagged-1 (188-204) TFA, JI130, Procyanidin B2, 3,3′-di-O-gallate, Limantrafin (CB-103), Tangeretin (Tangeritin), Carvacrol, RBPJ Inhibitor-1 (RIN1), IMR-1, Psoralidin, BMS-906024 or Tarlatamab (AMG-757).
Embodiment 15. The method of any one of embodiments 10-13, wherein the Notch1 inhibitor is a γ Secretase Inhibitor.
Embodiment 16. The method of embodiment 15, wherein the γ Secretase Inhibitor is DAPT (GSI-IX), LY-411575, LY900009, RO4929097 (RG-4733), YO-01027 (Dibenzazepine), Crenigacestat (LY3039478), Semagacestat, BMS-906024, Avagacestat (BMS-708163), or Z-Ile-Leu-aldehyde (Z-IL-CHO).
Embodiment 17. The method of any one of embodiments 10-13, wherein the Notch1 inhibitor is an anti-Notch1 antibody, a Notch1 decoy, CRISPR/CAS.
Embodiment 18. The method of embodiment 12, wherein sensitizing the cancer cells to an alternating electric field enhances the therapeutic efficacy of the alternating electric field.
Embodiment 19. The method of any of the preceding embodiments, wherein the alternating electric field is applied before, after, or simultaneously with administering the Notch1 inhibitor.
Embodiment 20. The method of any of the preceding embodiments, wherein the Notch1 inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.
Embodiment 21. The method of any one of embodiments 10-20, wherein the population of cells is in vitro.
Embodiment 22. The method of any one of embodiments 10-20, wherein the population of cells is in a subject.
Embodiment 23. The method of any of the preceding embodiments, wherein the alternating electric field has a frequency between 50 kHz and 1 MHz.
Embodiment 24. The method of any of the preceding embodiments, wherein the alternating electric field has a frequency of about 150 or 250 kHz.
Embodiment 25. The method of any of the preceding embodiments, wherein the alternating electric field has a field strength of between 0.5 and 10 V/cm RMS.
Embodiment 26. The method of any of the preceding embodiments, wherein the alternating electric field has a field strength of about 0.9 V/cm RMS.
Embodiment 27. The method of any of the preceding embodiments, further comprising administering a cancer therapeutic.
Embodiment 28. The method of any of the preceding embodiments, further comprising detecting an increase in Notch1 expression in the subject or cell after applying an alternating electric field and prior to administering a Notch1 inhibitor.
Embodiment 29. The method of any of the preceding embodiments, wherein administering a Notch1 inhibitor is performed 1, 2, 3, 4, 5, 6, 7, 8, 9, or 10 days after applying an alternating electric field is performed.
Embodiment 30. The method of any one of embodiments 11-29, wherein the population of cells comprises cancer cells.
Embodiment 31. The method of any one of embodiments 1-10, 13-17, or 19-30, wherein the target site comprises cancer cells.
Embodiment 32. The method of any one of embodiments 30 or 31, wherein the cancer cells are glioblastoma, ovarian cancer, non-small cell lung cancer, breast cancer (e.g. triple-negative breast cancer), papillary thyroid cancer, clear cell renal cell carcinoma, hepatocellular cancer, pleural mesothelioma, pancreatic cancer, lung cancer, gastric cancer, colon cancer or endometrial carcinoma.
Embodiment 33. The method of any of the preceding embodiments, wherein the subject has become desensitized to the alternating electric field.
Embodiment 34: The method of any of the preceding embodiments, wherein the Hes5 inhibitor is a Notch signaling inhibitor or DNA methylation inhibitor.
Embodiment 35: The method of embodiment 34, wherein the Notch signaling inhibitor is a β-secretase inhibitor, Notch receptor-blocking antibody, or Notch transcription complex-blocking peptide.

1. A method of treating a subject in need thereof comprising:
applying an alternating electric field to a target site of the subject in need thereof; and
administering a Notch1 inhibitor or Hes5 inhibitor to the subject in need thereof.

2. The method of claim 1, wherein the Notch1 inhibitor is a γ Secretase Inhibitor, anti-Notch1 antibody (mAb to DSL ligands, NRR antibodies, mAbs to Notch receptors, mAbs to Nicastrin, mAbs to ADAMS, mAbs to NICD, mAbs to MAM), a Notch1 decoy, Notch1 antagonist, siRNA, CRISPR/CAS, α-secretase inhibitor, acidification inhibitors, MAM stapled peptides.

3. The method of claim 3, wherein the γ Secretase Inhibitor is DAPT (GSI-IX), LY-411575, LY900009, RO4929097 (RG-4733), YO-01027 (Dibenzazepine), Crenigacestat (LY3039478), Semagacestat, BMS-906024, Avagacestat (BMS-708163), Sulindac sulfide, Dihydroergocristine mesylate (DHEC mesylate), γ-Secretase-IN-1, BMS 433796, L-685458, BMS 299897, MDL-28170, Nirogacestat, BT-GSI, E 2012, MK-0752, SPL-707, MRK-560, Itanapraced, JNJ-40418677, Z-Ile-Leu-aldehyde (Z-IL-CHO), or BT-GSI.

4. The method of claim 1, wherein the Notch1 inhibitor is, Jagged-1 (188-204) TFA, JI130, Procyanidin B2, 3,3′-di-O-gallate, Limantrafin (CB-103), Tangeretin (Tangeritin), Carvacrol, RBPJ Inhibitor-1 (RIN1), IMR-1, Psoralidin, BMS-906024 or Tarlatamab (AMG-757), Bruceine D, FLI-06, ZLDI-8, JI051, Rovalpituzumab, BMS-983970, BMS-986115, Brontictuzumab, IMR-1A, Tarextumab, Demcizumab, ASR-490, Navicixizumab, SAHM1, SAHM1 TFA, Enoticumab, FLI-06, SAHM1, NVS-ZP7-4, or JI051.

5. The method of claim 1, wherein the subject has cancer.

6. The method of claim 7, wherein the cancer is glioblastoma, ovarian cancer, non-small cell lung cancer, breast cancer (e.g. triple-negative breast cancer), papillary Thyroid cancer, clear cell renal cell carcinoma, hepatocellular cancer, pleural mesothelioma, pancreatic cancer, lung cancer, gastric cancer, colon cancer or endometrial carcinoma.

7. The method of claim 1, wherein the Notch1 inhibitor reduces cancer cell growth.

8. A method of reducing cancer cell growth comprising:
applying an alternating electric field to a population of cells comprising one or more cancer cells; and
contacting a Notch1 inhibitor or Hes5 inhibitor to the population of cells.

9. A method of sensitizing cancer cells to an alternating electric field comprising:
applying an alternating electric field to a population of cells comprising one or more cancer cells; and
contacting a Notch1 inhibitor or Hes5 inhibitor to the population of cells.

10. The method of claim 1, wherein the alternating electric field is applied before, after, or simultaneously with administering the Notch1 inhibitor.

11. The method of claim 1, wherein the Notch1 inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

12. The method of claim 1, wherein the alternating electric field has a frequency between 50 kHz and 1 MHz.

13. The method of claim 1, wherein the alternating electric field has a frequency of about 150 or 250 kHz.

14. The method of claim 1, wherein the alternating electric field has a field strength of between 0.5 and 10 V/cm RMS.

15. The method of claim 1, further comprising administering a cancer therapeutic.

16. The method of claim 1, further comprising detecting an increase in Notch1 expression in the subject or cell after applying an alternating electric field and prior to administering a Notch1 inhibitor.

17. The method of claim 1, wherein administering a Notch1 inhibitor is performed 1, 2, 3, 4, 5, 6, 7, 8, 9, or 10 days after applying an alternating electric field is performed.

18. The method of claim 1, wherein the population of cells comprises cancer cells.

19. The method of claim 1, wherein the target site comprises cancer cells.

20. The method of claim 1, wherein the Hes5 inhibitor is a Notch signaling inhibitor or DNA methylation inhibitor.
